(19) United States
(12) Patent Application Publication
(10) Pub. No.: US 2024/0327539 A1
(43) Pub. Date: Oct. 3, 2024

(54) METHODS OF EFFECTING A HEMODYNAMIC CHANGE BY ADMINISTERING AN ANTI-NPR1 ANTIBODY

(71) Applicant: REGENERON PHARMACEUTICALS, INC., Tarrytown, NY (US)

(72) Inventors: Michael E. DUNN, Montvale, NJ (US); Aaron KITHCART, New York, NY (US); Benjamin Adam OLENCHOCK, Ridgefield, CT (US)

(21) Appl. No.: 18/045,671

(22) Filed: Oct. 11, 2022

Related U.S. Application Data
(60) Provisional application No. 63/254,447, filed on Oct. 11, 2021.

(51) Int. Cl. C07K 16/40; A61K 39/395; A61P 9/12

(52) U.S. Cl. CPC C07K 16/40; A61K 39/3955; A61P 9/12; A61K 2039/505; A61K 2039/545; C07K 2317/565; C07K 2317/75

(57) ABSTRACT

The present disclosure is related to methods of reducing blood pressure or effecting a hemodynamic change in a subject, the methods comprising administering to the subject a pharmaceutical composition comprising an agonist of natriuretic peptide receptor 1 (NPR1) (e.g., an agonist anti-NPR1 antibody).

### CROSS-REFERENCED TO RELATED APPLICATIONS

This application claims priority to U.S. Provisional Patent Application No. 63/254,447, filed on Oct. 11, 2021, the entire contents of which is incorporated by reference herein.

### FIELD OF THE DISCLOSURE

The present disclosure is related to methods of treating a disease associated with altered hemodynamics, methods of reducing blood pressure, and methods of effecting a hemodynamic change in a subject, comprising administering to the subject a pharmaceutical composition comprising a therapeutically effective amount of an antibody or antigen-binding fragment thereof that specifically binds NPR1.

### REFERENCE TO A SEQUENCE LISTING XML

This application contains a Sequence Listing in XML format. The XML file is incorporated herein by reference. Said XML listing, created on Oct. 7, 2022, is named 40848_0111USU1_SL.xml and is 9,933 bytes in size.

### BACKGROUND

Natriuretic peptide receptor 1 (NPR1) is a membrane-bound guanylate cyclase that mediates the intracellular conversion of guanosine triphosphate to cyclic guanosine monophosphate (cGMP) (Martinez-Rumayor, et al., 2008 Am J Cardiol 101 (3a): 3-8). NPR1 is broadly expressed, including in the kidneys, vasculature, adrenal glands, and brain (Yancy, et al., 2017 Circulation 136 (6): e137-e161). NPR1 agonism results in alterations of systemic blood pressure (BP) through cGMP-mediated effects on intravascular volume, vasorelaxation, natriuresis, and diuresis.

NPR1 is activated by cardiac hormones atrial natriuretic peptide (ANP) and brain natriuretic peptide (BNP), which are synthesized as propeptides (proANP and proBNP) and stored by the heart within secretory granules located in the atrial and/or ventricular myocardium (Martinez Rumayor 2008). Upon release in response to stretch induced by increased pressure or neurohormonal stimuli, proANP and proBNP are enzymatically cleaved into biologically active ANP and BNP. Clearance of these peptides from circulation occurs primarily through degradation by matrix metalloproteinases including neprilysin, and via the natriuretic peptide clearance receptor, NPR3 (Martinez Rumayor 2008). Natriuretic peptides have important roles in the regulation of diuresis and natriuresis, along with inhibition of the renin-angiotensin-aldosterone system, with their effects culminating in modulation of local and systemic hemodynamics. Inhibition of neprilysin, a metalloprotease that degrades natriuretic peptides, decreases clearance of natriuretic peptides and has been studied in patients with heart failure. Neprilysin inhibition leads to an increase in endogenous ANP and BNP, which in turn stimulates NPR1 activity.

Monoclonal antibodies to NPR1 were first described by Kitano, et al., in 1995 (Immunol. Lett. 47:215-22). Activating or agonist anti-NPR1 antibodies are disclosed in, for example, U.S. Pat. No. 9,090,695, and 20160168251, and in WO2010065293. Fully human antibodies that specifically bind to NPR1 protein with high affinity and activate it could be important in the prevention and treatment of, e.g., hypertension, obesity, and heart failure.

### BRIEF SUMMARY

REGN5381 is a human immunoglobulin G4-based monoclonal antibody (mAb) that binds and activates NPR1 in both the presence and absence of the endogenous ligands ANP and BNP. Signaling through NPR1 directly may replicate many of the physiologic responses seen in other natriuretic pathway therapies. REGN5381 is a direct agonist of NPR1, whose long duration of effect may provide sustained hemodynamic control and cardiac unloading compared with current therapies, such as a combination of the neprilysin inhibitor, sacubitril and valsartan, or the recombinant BNP peptide nesiritide. As shown herein, REGN5381 induced predictable and persistent reductions of systemic BP in normotensive telemetered male cynomolgus monkeys (non-human primates (NHP)). Accordingly, REGN5381 is contemplated herein for treatment of diseases associated with altered hemodynamics including heart failure, hypertension, and chronic kidney disease.

Importantly, REGN5381 administration results in a decrease in blood pressure that is sustained relative to other known agents, without being accompanied by the possible negative effects typically associated with hypotension. As a result, the antibody or antigen-binding fragment thereof can be administered less frequently.

In one aspect, the disclosure provides a method of treating a disease associated with altered hemodynamics in a subject, comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1). In additional embodiments, the disease is selected from the group consisting of heart failure, hypertension, and chronic kidney disease.

In another aspect, the disclosure provides a method of reducing blood pressure in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1). In additional embodiments, the blood pressure is selected from the group consisting of systolic blood pressure, diastolic blood pressure, mean arterial pressure, and pulse pressure.

In still another aspect, the disclosure provides a method of effecting a hemodynamic change in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1). In additional embodiments, the hemodynamic change is a reduction in venous pressure, left ventricular end-diastolic pressure (LVEDP), and/or arterial pulse pressure (PP). In still further embodiments, the reduction in venous pressure is a reduction in central venous pressure (CVP).

In certain embodiments of a method according to the disclosure, urine output and systemic organ perfusion are not affected by the administration.

In certain embodiments of a method according to the disclosure, the heart rate (HR) of the subject increases.

In certain embodiments of a method according to the disclosure, the antibody or antigen-binding fragment thereof comprises three heavy chain complementarity determining regions (CDRs) (HCDR1, HCDR2 and HCDR3) contained within a heavy chain variable region (HCVR) comprising the amino acid of SEQ ID NO: 1; and three light chain CDRs (LCDR1, LCDR2 and LCDR3) contained within a light chain variable region (LCVR) comprising the amino acid sequence of SEQ ID NO: 2.

In certain embodiments of a method according to the disclosure, the antibody or antigen-binding fragment thereof comprises three heavy chain complementarity determining regions (CDRs) (HCDR1, HCDR2 and HCDR3) and three light chain CDRs (LCDR1, LCDR2 and LCDR3), wherein HCDR1 has the amino acid sequence of SEQ ID NO:3, HCDR2 has the amino acid sequence of SEQ ID NO:4, HCDR3 has the amino acid sequence of SEQ ID NO:5, LCDR 1 has the amino acid sequence of SEQ ID NO:6, LCDR2 has the amino acid sequence of VAS, and LCDR3 has the amino acid sequence of SEQ ID NO:8.

In certain embodiments of a method according to the disclosure, the antibody or antigen-binding fragment thereof comprises a heavy chain variable region (HCVR) comprising the amino acid of SEQ ID NO:1 and a light chain variable region (LCVR) comprising the amino acid sequence of SEQ ID NO:2.

In certain embodiments of a method according to the disclosure, the antibody comprises a heavy chain and a light chain, wherein the heavy chain comprises an amino acid sequence of SEQ ID NO: 9.

In certain embodiments of a method according to the disclosure, the antibody comprises a heavy chain and a light chain, wherein the light chain comprises an amino acid sequence of SEQ ID NO: 10.

In certain embodiments of a method according to the disclosure, the antibody comprises a heavy chain and a light chain, wherein the heavy chain comprises an amino acid sequence of SEQ ID NO: 9 and the light chain comprises an amino acid sequence of SEQ ID NO: 10.

In certain embodiments, the antibody or antigen-binding fragment thereof is administered at a low dose.

In certain embodiments of a method according to the disclosure, the antibody or antigen-binding fragment thereof is administered at a dose of about 0.031 to about 25 mg/kg of the body weight of the subject.

In certain embodiments of a method according to the disclosure, the antibody or antigen-binding fragment thereof is administered at a dose of about 1 mg to about 200 mg.

In certain embodiments of a method according to the disclosure, the antibody or antigen-binding fragment thereof is administered intravenously, subcutaneously, intradermally, intraperitoneally, or intramuscularly to the subject.

In certain embodiments of a method according to the disclosure, the antibody or antigen-binding fragment thereof is administered to the subject as a single dose.

In certain embodiments, the method according to the disclosure further comprises administering an additional/second therapeutic agent to the subject. In certain embodiments, the additional therapeutic agent may be selected from the group consisting of an aldosterone antagonist, an alpha-adrenergic blocker, an angiotensin converting enzyme (ACE) inhibitor, an arteriolar vasodilator, an autonomic ganglionic vasodilator, a beta-adrenergic blocker, a catecholamine-depleting sympatholytic, a central alpha-2 adrenergic agonist, a calcium channel blocker, a diuretic, a renin inhibitor, an anti-coagulant, an anti-platelet agent, a cholesterol lowering agent, a vasodilator, digitalis, surgery, an implantable device, anti-tumor therapy, insulin, a GLP1 agonist, metformin, dialysis, bone marrow stimulant, hemofiltration, a lifestyle modification, a dietary supplement, and any other drug or therapy known in the art. In certain embodiments, the additional therapeutic agent may be an agent that helps to counteract or reduce any possible side effect(s) associated with an antibody or antigen-binding fragment thereof that specifically binds NPR1, if such side effect(s) should occur. In certain embodiments, the additional therapeutic agent is administered concomitantly with or separately from the composition. In certain embodiments, the second therapeutic agent is administered concomitantly with or separately from the antibody or antigen-binding fragment thereof.

In certain embodiments of a method according to the disclosure, the subject is hypertensive. In one embodiment, the subject is mildly hypertensive. In one embodiment, the subject is human.

In one aspect, the disclosure provides a pharmaceutical composition for use in treating a disease associated with altered hemodynamics in a subject, comprising a therapeutically effective amount of an antibody or antigen-binding fragment thereof that specifically binds natriuretic peptide receptor 1 (NPR1) and a pharmaceutically acceptable carrier or diluent.

In another aspect, the disclosure provides a pharmaceutical composition for use in reducing blood pressure in a subject, comprising a therapeutically effective amount of an antibody or antigen-binding fragment thereof that specifically binds NPR1 and a pharmaceutically acceptable carrier or diluent.

In still another aspect, the disclosure provides a pharmaceutical composition for use in effecting hemodynamic changes in a subject, comprising a therapeutically effective amount of an antibody or antigen-binding fragment thereof that specifically binds natriuretic peptide receptor 1 (NPR1) and a pharmaceutically acceptable carrier or diluent.

In further aspects, the disclosure provides the use of an anti-NPR1 antibody or antigen-binding fragment thereof of the disclosure in the manufacture of a medicament for the treatment of a disease associated with altered hemodynamics in a subject, for the reduction of blood pressure in a subject, and/or for effecting hemodynamic changes in a subject.

Other embodiments will become apparent from a review of the ensuing detailed description.

### BRIEF DESCRIPTION OF THE FIGURES

FIGS. 1A-1E show the effects of REGN5381 on blood pressures and heart rate in normotensive NPR1^{hu/hu} mice, with (FIG. 1A) on systolic blood pressure, (FIG. 1B) on diastolic blood pressure, (FIG. 1C) on pulse pressure, (FIG. 1D) on heart rate, and (FIG. 1E) on mean arterial pressure. Telemetered normotensive NPR1^{hu/hu} mice were randomized into five groups of equal body weight and given a single subcutaneous injection of REGN5381 at the doses listed in Table 3. Phosphate-buffered saline (PBS) was used as control. All values are mean±SEM, n=3-6 per group.

FIG. 2 shows the Day 7 REGN5381 serum concentrations. Telemetered normotensive NPR1^{hu/hu} mice were randomized into five groups of equal body weight and given a single subcutaneous injection of REGN5381 at the doses listed in Table 1. Phosphate-buffered saline was used as control. Blood was collected on study day 7, and REGN5381 concentrations were assessed. All values are mean±SEM, n=5-6 per group.

FIGS. 3A-3D show the effects of low doses of REGN5381 on blood pressures and heart rate on telemetered normotensive Cynomolgus monkeys: (FIG. 3A) systolic blood pressure—change from baseline; (FIG. 3B) diastolic blood pressure—change from baseline; (FIG. 3C) heart rate—change from baseline; (FIG. 3D) mean arterial blood pressure—change from baseline. Telemetered normotensive cynomolgus monkeys were randomized into four groups of equal body weight and given a single intravenous injection of REGN5381 at the doses listed in Table 4. Systolic pressure, diastolic pressure, heart rate, and mean arterial pressure are graphically depicted from 43 hours prior to through 48 hours post-dose. Telemetry data are presented as 1-hour bins, with gray area representing the 12-hour dark cycle. All values are mean±SEM, n=4-7 per group.

FIGS. 4A and 4B show that REGN5381-associated acute reduction in central venous pressure correlates with a reflex tachycardia response. FIG. 4A shows the individual change from baseline central venous pressure; and FIG. 4B shows the correlation between changes in central venous pressure and heart rate. Male beagle dogs aged 11- to 13-months old were anesthetized and instrumented for cardiovascular hemodynamic data and urine collection. Animals each received a single IV bolus of saline (n=6), 10 mg/kg valsartan (n=4), or 25 mg/kg REGN5381 (n=6). The left-ventricular and systemic pressure waveforms were collected for each animal pre-dose (for baseline measurements) and during a 4-hour post-dose monitoring period. (FIG. 4A) Changes from baseline central venous pressure (CVP) for each individual animal over the first hour post-dose are shown; each line represents one individual animal. (FIG. 4B) The correlation between changes from baseline CVP and changes from baseline heart rate (HR) are depicted for each animal at the 15-minute post-dose timepoint; each symbol represents one individual animal.

FIG. 5 shows that REGN5381 induces an acute reduction in left ventricular end-diastolic pressure. Male beagle dogs aged 11- to 13-months old were anesthetized and instrumented for cardiovascular hemodynamic data and urine collection. Animals each received a single IV bolus of saline (n=6), 10 mg/kg valsartan (n=4), or 25 mg/kg REGN5381 (n=6). The left-ventricular and systemic pressure waveforms were collected for each animal pre-dose (for baseline measurements) and during a 4-hour post-dose monitoring period. Mean changes from baseline left ventricular end-diastolic pressure (LVEDP) for each treatment group over the first hour post-dose are shown. Data are expressed as the group mean±standard error of the mean.

FIGS. 6A and 6B show that REGN5381 does not induce changes in arterial pressures. FIG. 6A shows the mean change from baseline mean arterial pressure; and FIG. 6B shows the mean change from baseline arterial pulse pressure. Male beagle dogs aged 11- to 13-months old were anesthetized and instrumented for cardiovascular hemodynamic data and urine collection. Animals each received a single IV bolus of saline (n=6), 10 mg/kg valsartan (n=4), or 25 mg/kg REGN5381 (n=6). The left-ventricular and systemic pressure waveforms were collected for each animal pre-dose (for baseline measurements) and during a 4-hour post-dose monitoring period. (FIG. 6A) Mean changes from baseline mean arterial pressure (MAP) for each treatment group over the first hour post-dose are shown. (FIG. 6B) Mean changes from baseline arterial pulse pressure (PP) for each treatment group over the first hour post-dose are shown. Data are expressed as the group mean±standard error of the mean.

FIG. 7 shows a study flow diagram for Parts A and B of the study described in Example 5.

### DETAILED DESCRIPTION

Before the present methods are described, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are incorporated herein by reference in their entirety.

### Methods of Effecting Hemodynamic Changes

Disclosed herein is a method of treating a disease associated with altered hemodynamics in a subject, comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

As used herein, the terms “treat”, “treating”, or “treatment” refer to the reduction or amelioration of the severity of at least one symptom or indication of a disease or disorder associated with altered hemodynamics in a subject due to the administration of a therapeutic agent such as an antibody described herein to the subject in need thereof. The terms include inhibition of progression of disease or of worsening of a symptom/indication. The terms also include positive prognosis of disease, i.e., the subject may be free of disease or may have reduced disease upon administration of a therapeutic agent such as an antibody of the present disclosure. In one embodiment, the symptom or indication refers to blood pressure of the subject. The therapeutic agent may be administered at a therapeutic dose to the subject, as discussed further below.

Diseases associated with altered hemodynamics include, for example, cardiovascular disease, atherosclerotic carotid stenosis, heart failure, stroke, hypertension, and chronic kidney disease.

As used herein, the phrase “altered hemodynamics” refers to changes in blood flow. Changes in blood flow may include changes in one or more of heart rate, stroke volume, cardiac output, systemic vascular resistance, and blood pressure. In specific embodiments, the altered hemodynamics are in the form of a reduction in venous pressure, left ventricular end-diastolic pressure (LVEDP), and/or arterial pulse pressure (PP). The reduction in venous pressure may be a reduction in central venous pressure (CVP).

Also disclosed herein is a method of reducing blood pressure in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

As used herein, the phrase “blood pressure” may refer to any one of systolic blood pressure, diastolic blood pressure, mean arterial pressure (area under the arterial pressure/time curve, divided by the cardiac cycle duration), and pulse pressure (difference between systolic and diastolic pressures). Methods for measurement of blood pressure are known in the art. Blood pressure is measured in units of millimeters of mercury (mm Hg) and is usually expressed in terms of systolic (blood) pressure over diastolic (blood) pressure. Measurement methods include auscultatory, oscillometric, ultrasound, finger cuff methods. It can generally be measured, for example, using a digital blood pressure monitor or a sphygmomanometer.

Additionally disclosed herein is a method of effecting a hemodynamic change in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

As used herein, the phrase “hemodynamic change” refers to a change in blood flow.

Changes in blood flow may include changes in one or more of heart rate, stroke volume, cardiac output, systemic vascular resistance, and blood pressure. In specific embodiments, the hemodynamic change is a reduction in venous pressure, left ventricular end-diastolic pressure (LVEDP), and/or arterial pulse pressure (PP). The reduction in venous pressure may be a reduction in central venous pressure (CVP).

As used herein, the term “subject” refers to an animal, preferably a mammal, more preferably a human, in need of amelioration, prevention, and/or treatment of a NPR1-associated disease or disorder such as hypertension. The term includes human subjects who have or are at risk of having such a disease or disorder. In some embodiments, the subject is normotensive. In some embodiments, the subject is mildly hypertensive and otherwise healthy. In some embodiments, the subject is hypertensive and otherwise healthy. In some embodiments, the subject is hypertensive or mildly hypertensive.

Hypertension exists on a spectrum and is, in some embodiments, defined as a systolic blood pressure (SBP) ≥100 mm Hg and ≤140 mm Hg and a diastolic blood pressure (DBP)≥60 mm Hg and ≤90 mm Hg, and in some embodiments, defined as a systolic blood pressure (SBP) ≥130 mm Hg and ≤165 mm Hg and a diastolic blood pressure (DBP)≥60 mm Hg and ≤100 mm Hg. Subjects who are normotensive vs. mildly hypertensive vs. hypertensive are recognized by those skilled in the art.

In certain embodiments, the methods herein comprise administering a low dose of the anti-NPR1 antibody or antigen-binding fragment thereof. As used herein, a “low dose” refers to a dose of the antibody that is ≤5 mg/kg of the subject's body weight. In certain embodiments, the term refers to a dose of the antibody that is between 1 mg and 200 mg. In certain embodiments, the administration of such a low dose leads to lowering of the blood pressure that is sustained from 24 hours to up to 56 days in a subject in need thereof.

### Anti-NPR1 Antibodies and Antigen-binding Fragments Thereof

The methods disclosed herein comprise administering antibodies or antigen-binding fragments thereof that specifically bind NPR1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” NPR1, as used in the context of the present disclosure, includes antibodies that bind NPR1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human NPR1 may, however, have cross-reactivity to other antigens, such as NPR1 molecules from other (non-human) species. As disclosed herein, antibodies that bind human NPR1 also cross-react with monkey and dog NPR1.

The antibodies or antigen-binding fragments thereof that specifically bind NPR1, as described herein, are agonists of natriuretic peptide receptor 1 (NPR1) (e.g., are agonist anti-NPR1 antibodies). An “activating antibody” or an “agonist antibody”, as used herein (or an “antibody that increases or potentiates NPR1 activity” or “an antibody that stabilizes the activated conformation”), is intended to refer to an antibody whose binding to NPR1 results in activation of at least one biological activity of NPR1. For example, the methods herein comprises administering an agonist antibody that specifically binds NPR1 that may decrease systemic blood pressure upon administration to a subject in need thereof.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan, et al., (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos, et al., 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-NPR1 monoclonal antibodies described herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies employed in the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic biological properties, reduced immunogenicity, etc. Antibodies and antigen-binding fragments obtained in this general manner are encompassed within the present disclosure.

The present disclosure also employs fully human anti-NPR1 monoclonal antibodies comprising variants of any of the HCVR, LCVR, and/or CDR amino acid sequences disclosed herein having one or more conservative substitutions. For example, the present disclosure may employ anti-NPR1 antibodies having HCVR, LCVR, and/or CDR amino acid sequences with, e.g., 10 or fewer, 8 or fewer, 6 or fewer, 4 or fewer, etc., conservative amino acid substitutions relative to any of the HCVR, LCVR, and/or CDR amino acid sequences disclosed herein.

The term “human antibody”, or “fully human antibody”, as used herein, is intended to include antibodies having variable and constant regions derived from human germline immunoglobulin sequences. The human mAbs described herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, or “fully human antibody”, as used herein, is not intended to include mAbs in which CDR sequences derived from the germline of another mammalian species (e.g., mouse), have been grafted onto human FR sequences. The term includes antibodies that are recombinantly produced in a non-human mammal, or in cells of a non-human mammal. The term is not intended to include antibodies isolated from or generated in a human subject.

The term “recombinant”, as used herein, refers to antibodies or antigen-binding fragments thereof described herein, created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to NPR1 protein.

In specific embodiments, antibody or antibody fragments described herein may be conjugated to a moiety such a ligand or a therapeutic moiety (“immunoconjugate”), a second anti-NPR1 antibody, or any other therapeutic moiety useful for treating a NPR1-associated disease or disorder.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson, (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet, et al., (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul, et al., (1990) J. Mol. Biol. 215:403-410 and (1997) Nucleic Acids Res. 25:3389-3402, each of which is herein incorporated by reference.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to NPR1 protein. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (Vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

According to certain exemplary embodiments, the anti-NPR1 antibody, or antigen-binding fragment thereof comprises a heavy chain variable region (HCVR), light chain variable region (LCVR), and/or complementarity determining regions (CDRs) comprising the amino acid sequences of any of the anti-NPR1 antibodies set forth in US patent application publication 20200123263, which is hereby incorporated by reference in its entirety. In certain exemplary embodiments, the anti-NPR1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-NPR1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of VAS; and the LCDR3 comprises the amino acid sequence of SEQ ID NO: 8. In yet other embodiments, the anti-NPR1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-NPR1 antibody, wherein the antibody comprises a heavy chain comprising the amino acid sequence of SEQ ID NO: 9. In some embodiments, the anti-NPR1 antibody comprises a light chain comprising the amino acid sequence of SEQ ID NO: 10. An exemplary antibody comprising a heavy chain variable region comprising the amino acid sequence of SEQ ID NO: 1 and a light chain variable region comprising the amino acid sequence of SEQ ID NO: 2 is the fully human anti-NPR1 antibody known as known as REGN5381.

According to certain exemplary embodiments, the methods of the present disclosure comprise the use of REGN5381 or a bioequivalent thereof. As used herein, the term “bioequivalent” refers to anti-NPR1 antibodies or NPR1-binding proteins or fragments thereof that are pharmaceutical equivalents or pharmaceutical alternatives whose rate and/or extent of absorption do not show a significant difference with that of a reference antibody (e.g., REGN5381) when administered at the same molar dose under similar experimental conditions, either single dose or multiple dose. In the context of the present disclosure, the term “bioequivalent” includes antigen-binding proteins that bind to NPR1 and do not have clinically meaningful differences with REGN5381 with respect to safety, purity and/or potency.

According to certain embodiments of the present disclosure, the anti-human NPR1, or antigen-binding fragment thereof, comprises a HCVR having 90%, 95%, 97% or 98% sequence identity to SEQ ID NO: 1.

According to certain embodiments of the present disclosure, the anti-human NPR1, or antigen-binding fragment thereof, comprises a LCVR having 90%, 95%, 97% or 98% sequence identity to SEQ ID NO: 2.

According to certain embodiments of the present disclosure, the anti-human NPR1, or antigen-binding fragment thereof, comprises a HCVR comprising an amino acid sequence of SEQ ID NO: 1 having no more than 5 amino acid substitutions. According to certain embodiments of the present disclosure, the anti-human NPR1, or antigen-binding fragment thereof, comprises a LCVR comprising an amino acid sequence of SEQ ID NO: 2 having no more than 2 amino acid substitutions.

Sequence identity may be measured by methods known in the art (e.g., GAP, BESTFIT, and BLAST).

The present disclosure also includes use of anti-NPR1 antibodies or antigen-binding fragments thereof in methods to reduce blood pressure or effect hemodynamic changes, wherein the anti-NPR1 antibodies or antigen-binding fragments thereof comprise variants of any of the HCVR, LCVR and/or CDR amino acid sequences disclosed herein having one or more conservative amino acid substitutions. For example, the present disclosure includes use of anti-NPR1 antibodies or antigen-binding fragments thereof having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-NPR1 antibodies or antigen-binding fragments thereof that can be used in the context of the methods of the present disclosure include, e.g., any of the anti-NPR1 antibodies set forth, for example, in US2012/0114659A1. The portions of all of the aforementioned publications that identify anti-NPR1 antibodies are hereby incorporated by reference.

### Therapeutic Administration and Formulations

The methods described herein comprise administrating anti-NPR1 antibody or antigen-binding fragment thereof to a subject in need thereof. The anti-NPR1 antibody or antigen-binding fragment thereof may be comprised in a pharmaceutical composition. Pharmaceutical (or “therapeutic”) compositions in accordance with the disclosure are administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations” PDA (1998) J Pharm Sci Technol 52:238-311.

The dose of antibody may vary depending upon the age and the size of a subject to be administered, target disease, conditions, route of administration, and the like. Depending on the severity of the condition, the frequency and the duration of the treatment can be adjusted. In certain embodiments, the antibody or antigen-binding fragment thereof can be administered as a single dose. In other embodiments, the antibody or antigen-binding fragment thereof can be administered as an initial dose followed by a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks. In certain embodiments, the subsequent dose(s) is/are administered at least 2 weeks, at least 3 weeks, or at least 4 weeks after the initial dose. In further embodiments, the drop in blood pressure resulting from administration of the anti-NPR1 antibody or antigen-binding fragment thereof is sustained between administration of the initial and subsequent doses.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu, et al., (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies described herein is also contemplated herein. Antibody-conjugated nanoparticles may be used both for therapeutic and diagnostic applications. Antibody-conjugated nanoparticles and methods of preparation and use are described in detail by Arruebo, M., et al., 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target cells. Nanoparticles for drug delivery have also been described in, for example, U.S. Pat. Nos. 8,257,740, or 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg or about 1 mg to about 200 mg per dosage form in a unit dose, especially in the form of injection.

### Combination Therapies

The methods of the present disclosure include administering an anti-NPR1 antibody or antigen-binding fragment thereof in combination with an additional therapeutic agent or therapy. Combination therapies may include an antibody or a biologically active fragment of an antibody described herein and any additional therapeutic agent that may be advantageously combined with the same.

Depending upon the disease, disorder or condition, the antibodies described herein may be used in combination with one or more additional therapeutic agents including, but not limited to, an aldosterone antagonist (e.g., eplerenone, spironolactone), an alpha-adrenergic blocker (e.g., doxazosin, phenoxybenzamine, phentolamine, prazosin, terazosin), an angiotensin converting enzyme (ACE) inhibitor (e.g., benazepril, captopril, enalapril, fosinopril, lisinopril, moexipril, perindopril, quinapril, ramipril, trandolapril), an arteriolar vasodilator (e.g., hydralazine, minoxidil), an autonomic ganglionic vasodilator (e.g., mecamylamine), a beta-adrenergic blocker (acebutolol, atenolol, betaxolol, bisoprolol, carvedilol, carteolol, esmolol, labetolol, metoprolol, nadolol, penbuterol, pindolol, propranolol, timolol), a catecholamine-depleting sympatholytic (e.g., deserpidine, reserpine), a central alpha-2 adrenergic agonist (e.g., clonidine, guanabenz, guanfacine, methyldopa), a calcium channel blocker (diltiazem, verapamil, amlodipine, felodipine, isradipine, nicadipine, nifedipine, nisoldipine), a diuretic (e.g., bumetanide, ethacrynic acid, furoseamide, torsemide, chlorothiazide, hydrochlorothiazide, hydroflumethiazide, methyclothiazide, polythiazide, chlorthalidone, indapamide, metolazone), a renin inhibitor (e.g., aliskiren), an anti-coagulant (e.g., coumardin, dabigatran, apixaban), an anti-platelet agent (e.g., aspirin, clopidogrel), a cholesterol lowering agent (e.g., a statin, a PCSK9 inhibitor such as alirocumab), a vasodilator (e.g., minoxidil, hydralazine, nitrates), digitalis, surgery (e.g., angioplasty, coronary artery bypass, heart transplant), an implantable device (e.g., valve replacement, defibrillator device, left ventricular assist device, pacemaker), anti-tumor therapy (e.g., a chemotherapeutic agent, surgery, radiation, a PD-1 inhibitor), insulin, a GLP1 agonist (e.g., exenatide, liraglutide, lixisenatide, albiglutide, dulaglutide, semaglutide), metformin, an agent that increases blood pressure, dialysis, bone marrow stimulant, hemofiltration, a lifestyle modification, and a dietary supplement.

As used herein, the term “in combination with” means that additional therapeutically active component(s) may be administered prior to, concurrent with, or after the administration of the anti-NPR1 antibody or antigen-binding fragment thereof. The term “in combination with” also includes sequential or concomitant administration of an anti-NPR1 antibody and a second therapeutic agent.

The additional therapeutically active component(s) may be administered to a subject prior to administration of an anti-NPR1 antibody or antigen-binding fragment thereof. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, or less than 30 minutes before administration of the second component. In other embodiments, the additional therapeutically active component(s) may be administered to a subject after administration of an anti-NPR1 antibody of the present disclosure. For example, a first component may be deemed to be administered “after” a second component if the first component is administered 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after or more after administration of the second component. In yet other embodiments, the additional therapeutically active component(s) may be administered to a subject concurrent with administration of an anti-NPR1 antibody or antigen-binding fragment thereof. “Concurrent” administration, for purposes of the present disclosure, includes, e.g., administration of an anti-NPR1 antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-NPR1 antibody and the additional therapeutically active component may be administered intravenously, etc.); alternatively, each dosage form may be administered via a different route (e.g., the anti-NPR1 antibody may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-NPR1 antibody “prior to”, “concurrent with,” or “after” (as those terms are defined herein above) administration of an additional therapeutically active component is considered administration of an anti-NPR1 antibody “in combination with” an additional therapeutically active component.

### Dosage

The amount of the anti-NPR1 antibody or antigen-binding fragment thereof administered to a subject according to the methods disclosed herein is, generally, a therapeutically effective amount. The phrase “therapeutically effective amount”, as used herein, refers to an amount of an antibody or antigen-binding fragment thereof that specifically binds natriuretic peptide receptor 1 (NPR1) that produces the desired effect for which it is administered. In certain embodiments, the desired effect is to lower blood pressure in a subject. In additional embodiments, the desired effect is to lower blood pressure in a subject without resulting in adverse effects associated with hypotension (including, without limitation, nausea, lightheadedness, vomiting, inadequate oxygen, heart problems, kidney damage, syncope (and associated injury), altered locomotion, and even death.

In certain embodiments, dosages can be expressed as flat doses or weight-based doses.

In certain embodiments, a therapeutically effective amount of the anti-NPR1 antibody or antigen-binding fragment thereof, such as REGN5381 or a bioequivalent thereof) can be from about 0.01 mg to about 500 mg, from about 0.1 mg to about 400 mg, from about 1 mg to about 300 mg, from about 5 mg to about 250 mg, from about 10 mg to about 200 mg, or from about 5 mg to about 150 mg of the antibody. For example, in various embodiments, the amount of the antibody is about 0.01 mg, about 0.05 mg, about 0.1 mg, about 0.2 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, or about 500 mg. In specific embodiments, the antibody or antigen-binding fragment thereof is administered at a low dose of about 1 mg to about 200 mg.

The amount of the antibody contained within an individual dose may be expressed in terms of milligrams of antibody per kilogram of subject body weight (i.e., mg/kg). In certain embodiments, the antibody used in the methods disclosed herein may be administered to a subject at a dose of about 0.0001 to about 50 mg/kg of subject body weight. In certain embodiments, an anti-NPR1 antibody may be administered at dose of about 0.1 mg/kg to about 25 mg/kg of a subject's body weight. In certain embodiments, the methods of the present disclosure comprise administration of the antibody at a dose of about 0.1 mg/kg to 5 mg/kg, 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a subject's body weight. In specific embodiments, the antibody or antigen-binding fragment thereof is administered at a dose of about 0.031 to about 5 mg/kg, or about 5 mg/kg to about 25 mg/kg of the body weight of the subject. In additional embodiments, a low dose of antibody or antigen-binding fragment is administered to the subject. In one embodiment, the low dose is ≤ about 5 mg/kg.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

The exemplary antibody used in the following examples is REGN5381, a fully human agonist antibody that specifically binds to NPR1 and comprises the HCVR/LCVR of SEQ ID NOS: 1/2, HCDR1-HCDR2-HCDR3-LCDR1-LCDR2-LCDR3 of SEQ ID NO:3-SEQ ID NO:4-SEQ ID NO:5-SEQ ID NO:6-VAS-SEQ ID NO:8. See also US 20200123263.

### Example 1: Effect on Systemic Blood Pressure of a Single Dose of an NPR1 Agonist mAb (R5381) in Normotensive NPR1^{hu/hu} Mice

### Experimental Procedure

In order to assess a dose response using low doses of the NPR1 agonist antibody REGN5381 on systemic blood pressure in telemetered normotensive NPR1^{hu/hu} mice, male NPR1^{hu/hu} mice (n=30) aged ˜11 weeks were implanted with PA-C10 telemeters (DSI, St. Paul, MN) and allowed to recover for at least 7 days, prior to being assigned to group (Groups 1-5) (Table 1). Animals were individually housed under standard conditions (Temperatures of 64° F. to 84° F. (18° C. to 29° C.); relative humidity of 30% to 70%), and a 12-hour light/12-hour dark cycle was maintained. Food (Research Diets Standard pellet chow) and water were provided ad libitum.

TABLE 1
Summary of Doses and Dose Groups

| | | | Dose | Number of |
|---|---|---|---|---|
| | | Dose Level | Volume | Animals |
| Group No. | Test or Control Article | (mg/kg/dose) | (mL/kg) | Males |
| 1 | PBS | 0 | 5 | 4 |
| 2 | REGN5381 | 0.125 | 5 | 7 |
| 3 | REGN5381 | 0.25 | 5 | 7 |
| 4 | REGN5381 | 0.5 | 5 | 7 |
| 5 | REGN5381 | 25 | 5 | 5 |

The test proteins were administered to the appropriate animals once on day 0 via subcutaneous injection. The dose volume for each animal was based on the most recent body weight measurement. Blood samples were collected on study day 7 and at termination for serum biomarker assessment, and urine was collected on day 22 for urine biomarker analysis.

Systolic pressure, diastolic pressure, mean arterial pressure, pulse pressure, and heart rate were collected for 10 seconds every 10 minutes for the duration of the testing period. Graphically displayed telemetry data were obtained from animals with viable signals for the duration of the in-life portion of the study.

### Results

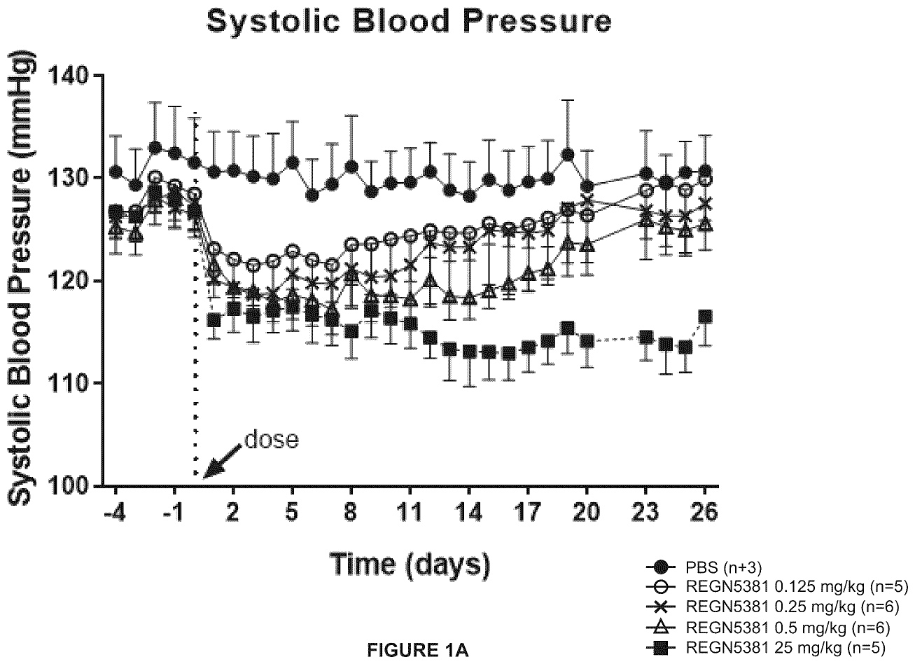

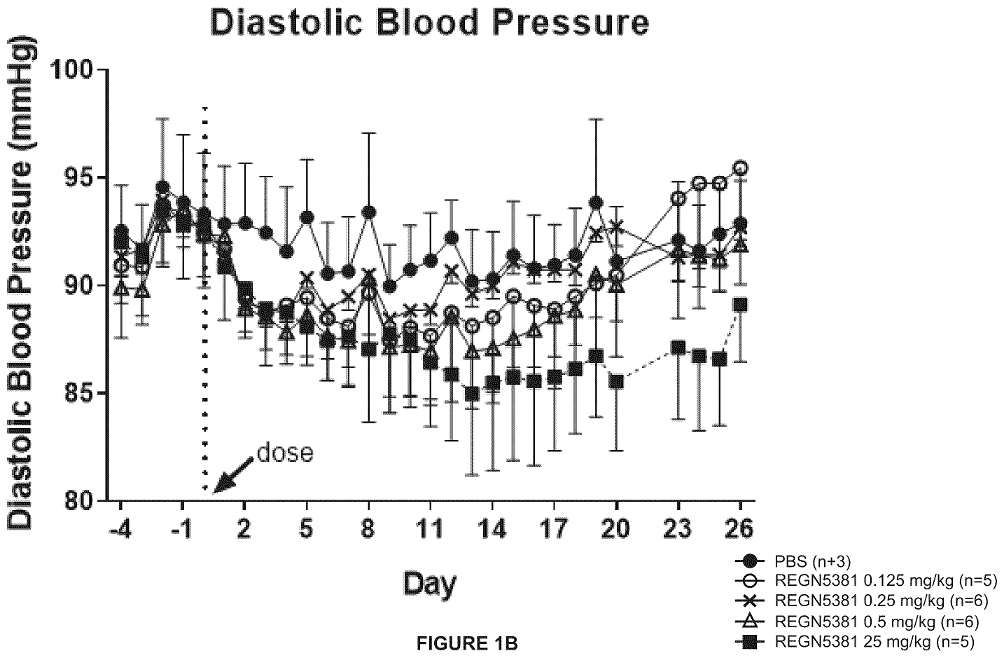

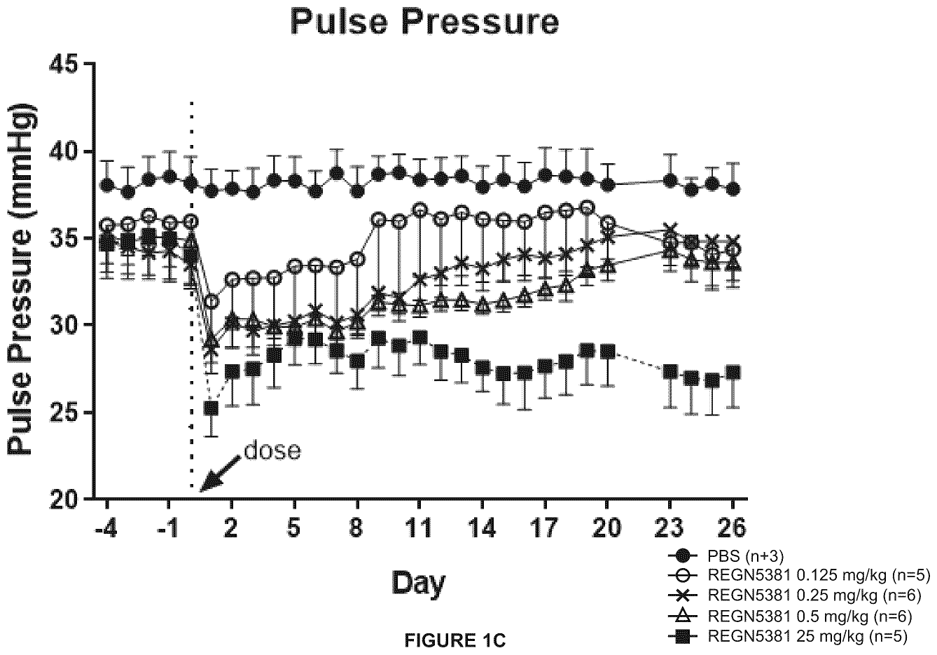

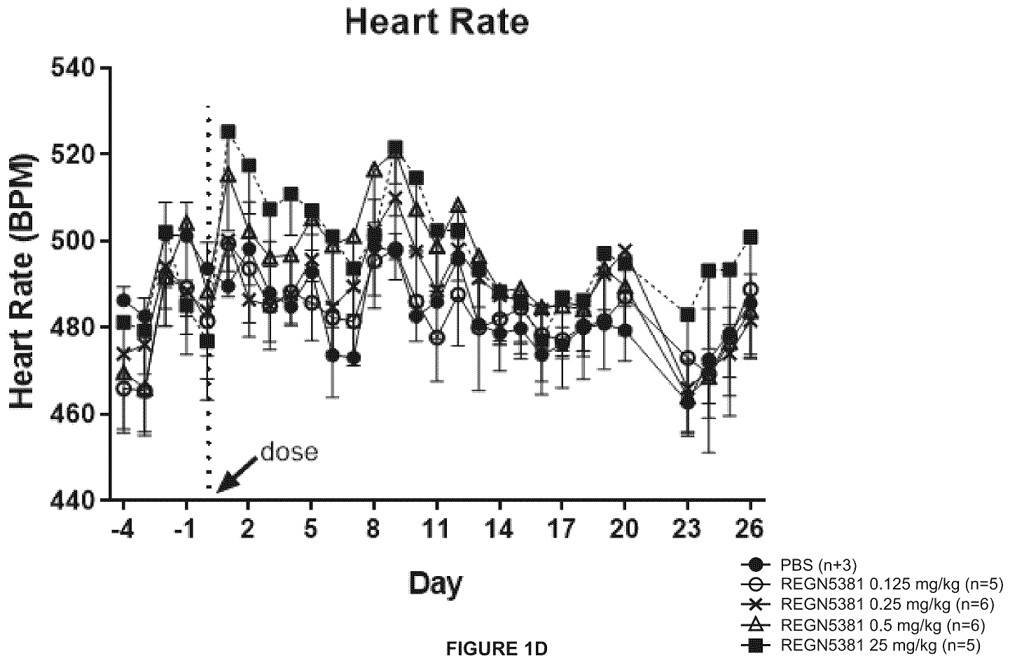

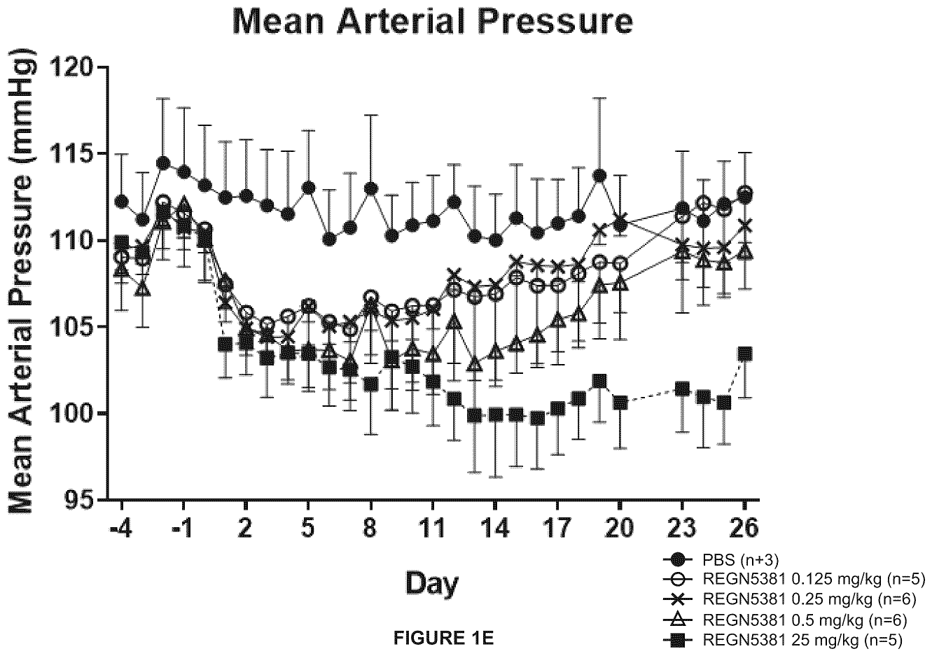

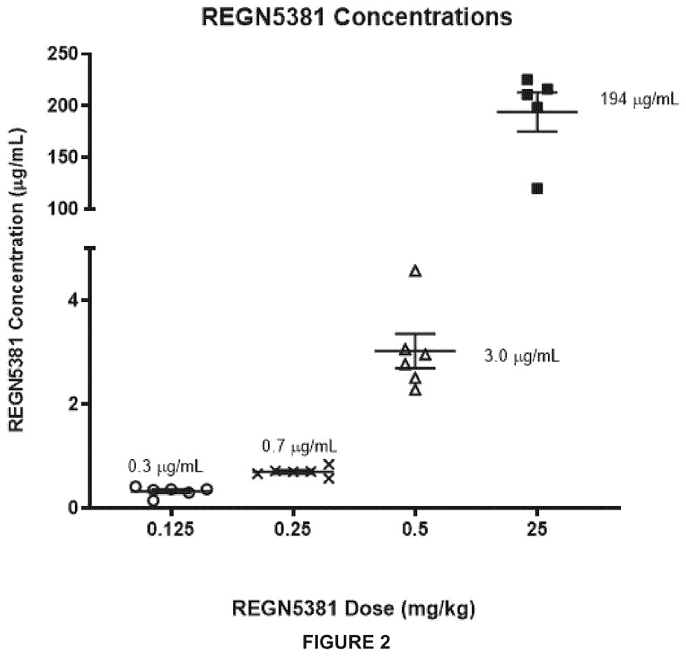

Blood pressures (FIGS. 1A-1C and 1E) were significantly and persistently reduced in all normotensive NPR1^{hu/hu} mice that received a single dose of REGN5381. The magnitude of pressure reduction was largely dose-dependent, with the highest dose (25 mg/kg) resulting in approximate 10-15 mm Hg pressure reduction for the duration of the study. FIG. 1 shows changes in (1A) systolic, (1B) diastolic, (1C) pulse pressure, (1D) heart rate, and (1E) mean arterial pressures, following administration of a single dose of an NPR1 agonist mAb, REGN5381, or PBS. REGN5381 produced significant reductions in systolic, diastolic, pulse, and mean arterial blood pressures (FIG. 1, Table 2). The duration of pharmacodynamic effect was commensurate with dose, with the highest dose of REGN5381 (25 mg/kg) lasting up to 26 days. A compensatory increase in heart rate was observed in doses of 0.5 mg/kg or greater compared to PBS control dosed animals. Significant and persistent blood pressure effects were detected at single doses of REGN5381 as low as 0.125 mg/kg. The duration of pressure reduction was also dose-dependent and agreed with the Day 7 REGN5381 serum concentrations (FIG. 2), in that the highest exposures correlated with the longest test article-related effect on hemodynamic endpoints. Peak acute pressure reductions appeared to be dose-dependent, with the duration of blood pressure effect being approximately 10 days at 0.125 mg/kg and longer than 26 days for 25 mg/kg. No effect on urine volume was noted on day 22, and urinary cGMP concentrations (Table 3) assessed on day 22 were not significantly different from control values for any of the REGN5381 groups.

TABLE 2
26-day Mean Blood Pressures and Heart Rate

| | | | Mean | Pulse | Heart |
|---|---|---|---|---|---|
| | Systolic | Diastolic | Arterial | Pressure | Rate |
| Dose Group: | (mm Hg) | (mm Hg) | (mm Hg) | (mm Hg) | (BPM) |
| PBS | 130 ± 0.2 | 92 ± 0.5 | 112 ± 0.2 | 38 ± 0.1 | 483 ± 2 |
| REGN5381 | 125 ± 0.5**** | 90 ± 0.5** | 108 ± 0.5**** | 35 ± 0.3**** | 484 ± 1 |
| (0.125 mg/kg) | | | | | |
| REGN5381 | 123 ± 0.6**** | 90 ± 0.3* | 108 ± 0.4**** | 33 ± 0.4**** | 488 ± 2 |
| (0.25 mg/kg) | | | | | |
| REGN5381 (0.5 | 121 ± 0.6**** | 89 ± 0.3**** | 106 ± 0.4**** | 32 ± 0.3**** | 494 ± 3** |
| mg/kg) | | | | | |
| REGN5381 (25 | 116 ± 0.6**** | 87 ± 0.4**** | 102.8 ± 0.4**** | 28 ± 0.3**** | 498 ± 3**** |
| mg/kg) | | | | | |

Telemetered normotensive NPR1^{hu/hu} mice were randomized into five groups of equal body weight and given a single subcutaneous injection of REGN5381 at the doses listed in Table 1. Phosphate buffered saline (PBS) was used as a control. All values are mean ± SEM, n = 3-6 per group. Statistics - one-way ANOVA with Dunnett's;
*p < .05 vs. PBS,
**p < .01 vs. PBS;
****P < .0001 vs. PBS

TABLE 3
Day 22 Urine Volumes and Urinary cGMP Levels

| | Urine | Urinary | |
|---|---|---|---|
| | Volume | cGMP | Urinary cGMP |
| Dose Group: | (mL/day) | (pmol/mL) | (pmol/day) |
| PBS | 1.3 ± 0.1 | 4560 ± 563 | 5905 ± 952 |
| REGN5381 (0.125 | 1.4 ± 0.2 | 3897 ± 598 | 5480 ± 1093 |
| mg/kg) | | | |
| REGN5381 (0.25 | 1.4 ± 0.2 | 3756 ± 196 | 5150 ± 907 |
| mg/kg) | | | |
| REGN5381 (0.5 mg/kg) | 1.1 ± 0.2 | 3554 ± 482 | 4135 ± 1026 |
| REGN5381 (25 mg/kg) | 1.3 ± 0.1 | 6366 ± 983 | 8278 ± 1941 |

Telemetered normotensive NPR1^{hu/hu} mice were randomized into five groups of equal body weight and given a single subcutaneous injection of REGN5381 at the doses listed in Table 1. Phosphate-buffered saline (PBS) was used as a control. Urine was collected overnight beginning on study day 21 and ending on study day 22. All values are mean ± SEM, n = 4-6 per group. Statistics - one-way ANOVA with Dunnett's

### Example 2. Interrogation of a Single Intravenous Low Dose of REGN5381 on Systemic Blood Pressure in Telemetered Normotensive Cynomolgus Monkeys

### Experimental Procedure

In order to identify a low intravenous dose of the NPR1 agonist antibody REGN5381 that induced a transient but detectable reduction of systemic blood pressure in telemetered normotensive cynomolgus monkeys, male cynomolgus monkeys (n=22) weighing 3-5 kg were previously implanted with PhysioTel Digital model L11 telemeters (DSI, St. Paul, MN). Animals were acclimated to laboratory housing for at least 5 weeks prior to random assignment to dose group (Groups 1-4) (Table 4). Animals were housed under standard conditions (Temperatures of 64° F. to 84° F. (18° C. to 29° C.); relative humidity of 30% to 70%) and a 12-hour light/12-hour dark cycle was maintained. Food (PMI Nutrition International Certified Primate Chow No. 5048) and water were provided ad libitum.

TABLE 4
Summary of Doses and Dose Groups

| | | | Dose | Number of |
|---|---|---|---|---|
| | | Dose Level | Volume | Animals |
| Group No. | Test or Control Article | (μg/kg) | (mL/kg) | Males |
| 1 | Saline | 0 | 1 | 3 |
| 2 | REGN5381 | 8 | | 7 |
| 3 | | 31 | | 7 |
| 4 | | 125 | | 5 |

The test protein or saline control was administered to the appropriate animals once on day 1 via intravenous injection. The dose volume for each animal was based on the most recent body weight measurement. Blood samples for drug level assessment were collected at baseline, and 72 hours post-dose and on study days 7 and 14.

Systolic pressure, diastolic pressure, mean arterial pressure, and heart rate were collected continuously for the duration of the experiment, with data binned at 60-minute averages for the duration of the testing period. Telemetry data were obtained from animals with viable signals for the duration of the in-life portion of the study.

### Results

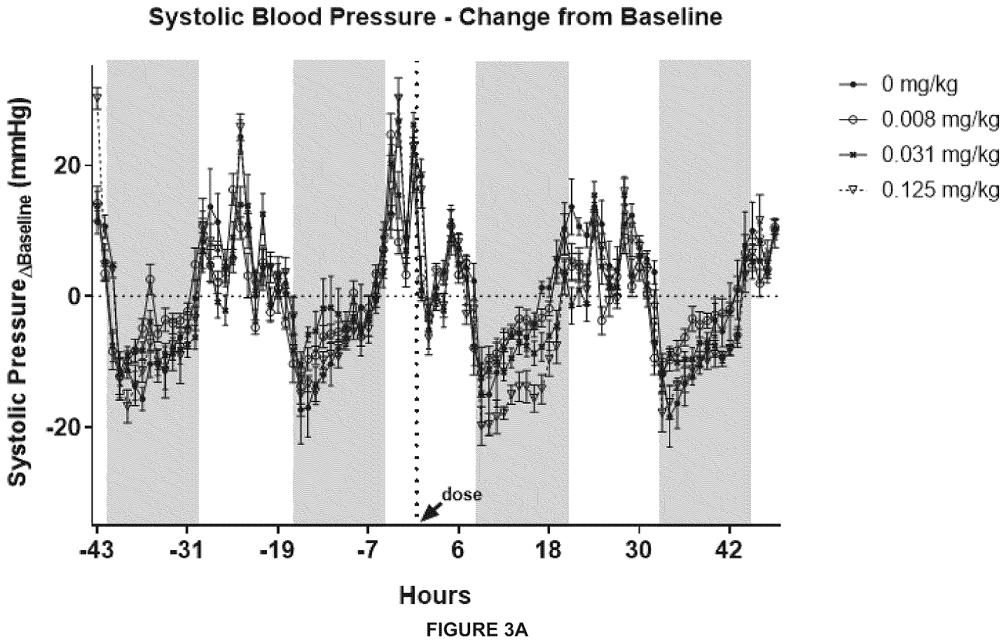

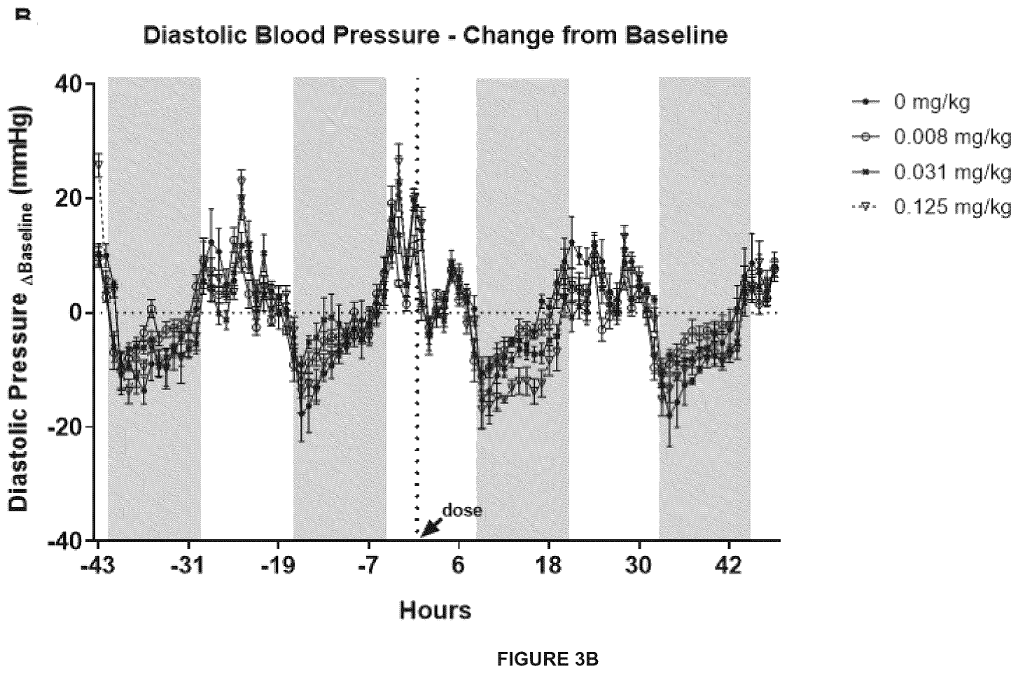

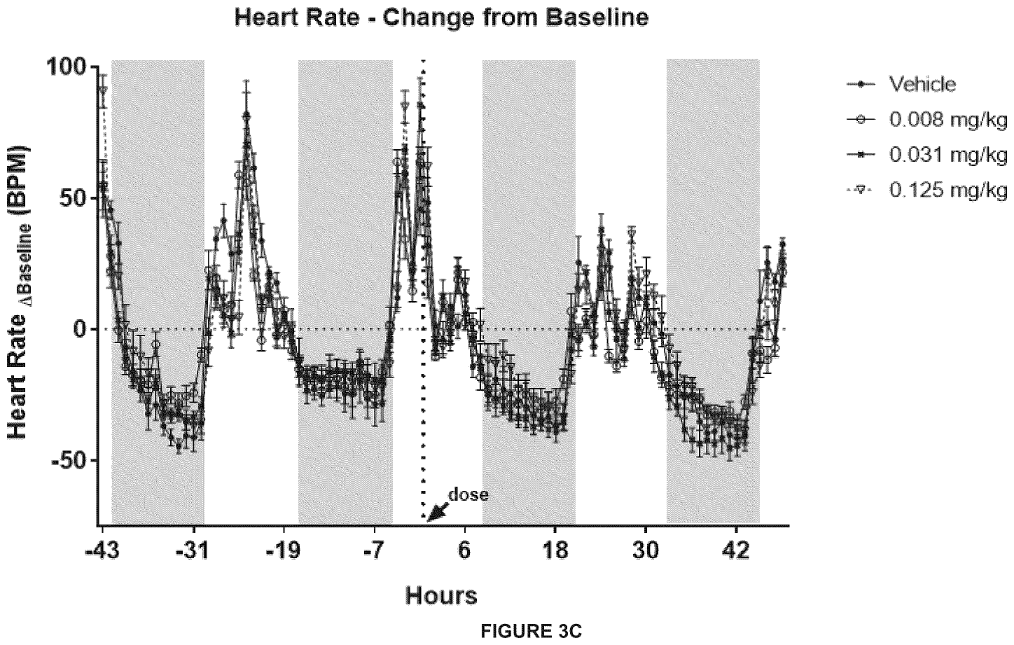

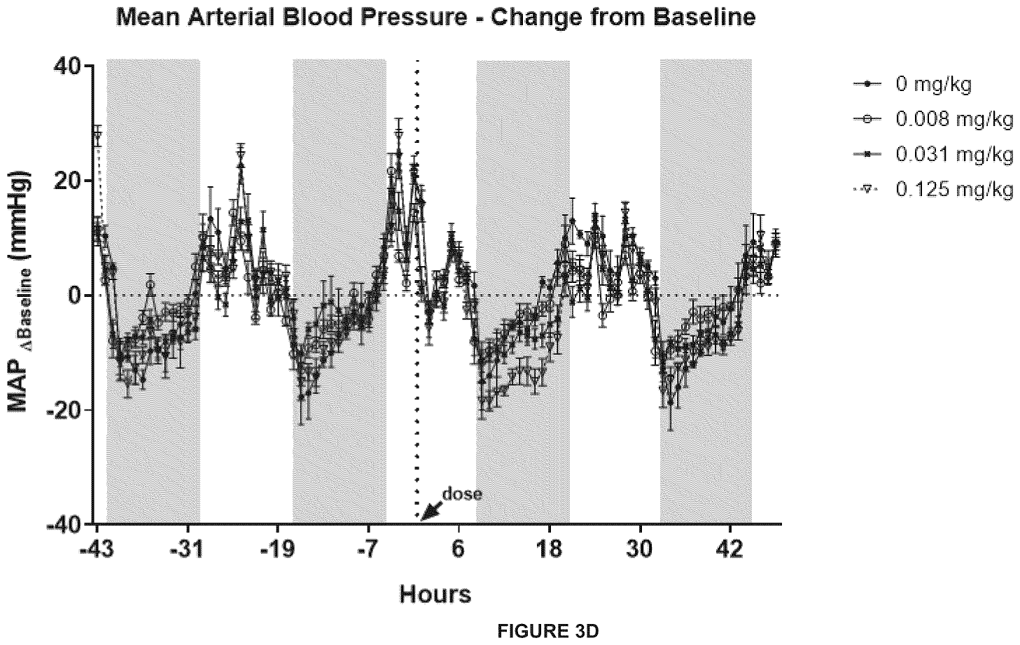

Blood pressures (Tables 5 and 6; FIGS. 3A, 3B, and 3D) were reduced in a dose-dependent manner over the first 24 hours. Statistically significant reductions in systolic pressure were observed over the first 24 hours post-dose in the 31 and 125 μg/kg groups. Systolic pressures were reduced approximately 3.0 and 5.1 mm Hg, respectively. These effects were transient, with pressures returning toward saline control levels over the 24- through 48-hour post-dose period. Statistically significant reductions in diastolic pressure were observed over the first 24 hours post-dose in the 31 and 125 μg/kg groups. Diastolic pressures were reduced approximately 2.6 and 4.2 mm Hg, respectively. These effects were transient, with pressures returning toward saline control levels over the 24 through 48-hour post-dose period. Consistent with the systolic and diastolic pressure effects, statistically significant reductions in mean arterial pressures (MAP) were observed over the first 24 hours post-dose in the 31 and 125 μg/kg groups. MAP's were reduced approximately 2.7 and 4.8 mm Hg, respectively. These effects were transient, with pressures returning toward saline control levels over the 24- through 48-hour post-dose period. The low dose of 8 μg/kg induced trending but non-statistically significant reductions of systolic, diastolic, and MAP over the first 24 hours, with no difference noted thereafter. Heart rates were reduced in all animals post-dose. No statistically significant REGN5381-related effects on heart rate were observed at any of the timepoints assessed, though a trending but non-statistically significant increase was noted in the 125 μg/kg when compared to saline control.

TABLE 5
Mean Systolic and Diastolic Pressures

| | Systolic Blood Pressure | | Diastolic Blood Pressure | |
|---|---|---|---|---|
| | (change from baseline) | | (change from baseline) | |
| Dose Group: | 0-24-hr mean | 24-48-hr mean | 0-24-hr mean | 24-48-hr mean |
| Saline | 1.64 ± 0.55 | −0.06 ± 0.55 | 1.0 ± 0.15 | −0.90 ± 0.45 |
| REGN5381 (8 μg/kg) | −0.16 ± 0.66 | −0.85 ± 0.89 | −0.60 ± 0.54 | −1.29 ± 0.76 |
| REGN5381 (31 μg/kg) | −3.07 ± 1.01** | −1.57 ± 0.60 | −2.63 ± 0.87* | −1.85 ± 0.52 |
| REGN5381 (125 μg/kg) | −5.08 ± 0.76*** | −1.78 ± 0.76 | −4.17 ± 0.76** | −1.63 ± 1.06 |

Telemetered normotensive cynomolgus monkeys were randomized into four groups of equal body weight and given a single intravenous injection of REGN5381 at the doses listed in Table 4. All values are mean ± SEM, n = 3-7 per group. Statistics - one way ANOVA with Dunnett's;
*p < .05,
**p < .01,
***p < .001 vs. saline control.

TABLE 6
Mean Heart Rate and Mean Arterial Blood Pressure

| | Heart Rate | | Mean Arterial Blood Pressure | |
|---|---|---|---|---|
| | (change from baseline) | | (change from baseline) | |
| Dose Group: | 0-24-hr mean | 24-48-hr mean | 0-24-hr mean | 24-48-hr mean |
| Saline | −9.40 ± 1.86 | −9.72 ± 2.59 | 1.5 ± 0.34 | −0.38 ± 0.48 |
| REGN5381 (8 μg/kg) | −10.08 ± 2.78 | −15.76 ± 2.23 | −0.29 ± 0.54 | −0.98 ± 0.79 |
| REGN5381 (31 μg/kg) | −12.02 ± 1.45 | −17.31 ± 2.07 | −2.73 ± 0.99* | −1.57 ± 0.59 |
| REGN5381 (125 μg/kg) | −4.95 ± 2.22 | −7.47 ± 2.28 | −4.75 ± 0.72*** | −1.61 ± 0.85 |

Telemetered normotensive cynomolgus monkeys were randomized into four groups of equal body weight and given a single intravenous injection of REGN5381 at the doses listed in Table 4. All values are mean ± SEM, n = 3-7 per group. Statistics - one way ANOVA with Dunnett's;
*p < .05,
**p < .01,
***p < .001 vs. saline control.

Total REGN5381 serum concentrations were measured using an enzyme-linked immunosorbent assay. Serum samples were obtained pre-dose, then 72 hours and 7 and 14 days post-dose. REGN5381 concentrations were below the limit of quantification of 0.078 μg/mL in all samples except the 125 μg/kg group at 72 hours post-dose.

TABLE 7
REGN5381 Serum Levels

| | Group 1 | Group 2 | Group 3 | Group 4 |
|---|---|---|---|---|
| Time Point: | μg/mL | μg/mL | μg/mL | μg/mL |
| Predose | BLQ | BLQ | BLQ | BLQ |
| 72 hours post-dose | BLQ | BLQ | BLQ | 0.31 ± 0.11 |
| Day 7 | BLQ | BLQ | BLQ | BLQ |
| Day 14 | BLQ | BLQ | BLQ | BLQ |

Telemetered normotensive cynomolgus monkeys were randomized into four groups of equal body weight and given a single intravenous injection of REGN5381 at the doses listed in Table 4. All values are mean ± SD, n = 3-7 per group. BLQ = Below Limit of Quantitation (<0.078 μg/mL in neat serum)

FIG. 3A shows changes in systolic pressures following administration of a single dose of an NPR1 agonist mAb, REGN5381 NPR1 agonist mAb, or saline. FIG. 3B shows changes in diastolic pressures following administration of a single dose of an NPR1 agonist mAb, REGN5381 NPR1 agonist mAb, or saline. FIG. 3C shows changes in heart rate following administration of a single dose of an NPR1 agonist mAb, REGN5381, or saline. FIG. 3D shows changes in mean arterial pressures following administration of a single dose of an NPR1 agonist mAb, REGN5381 NPR1 agonist mAb, or saline. The key findings are that the NPR1 agonist mAb, REGN5381 produced statistically significant but transient effects on blood pressure following single intravenous doses as low as 31 μg/kg. These effects were dose dependent, and persisted for less than 48 hours, as evidenced by a lack of a statistically significant difference in any of the REGN5381-dosed animals versus control over the 24 through 48-hour post-dose period. These pressure effects occurred independent of a significant effect on heart rate.

Further data shows the effect of REGN5381 on plasma volume (versus 5% blood draw down or control). Change in plasma volume over time was measured in 24 male cynomolgus monkeys assigned to one of three groups on day 0 and dosed on day 6 with saline (control, n=8), saline+5% blood drawdown (n=8), or REGN5381 at 25 mg/kg (n=8) via a cephalic vein or saphenous vein. On day 0 (baseline), day 6 (dosing), and day 20 (recovery), blood samples of 3-5 ml were collected from all animals for measurements of blood volume. Samples were analyzed for total radioactivity. Data are expressed as the group mean±standard error of the mean. Statistical significance was determined using a two-way repeated measure ANOVA followed with Tukey's post-hoc multiple comparisons test (α=0.05).

TABLE 8
Effect of REGN5381 on mean plasma volume
Mean Plasma Volume (mL)

| | Baseline | 4-hr post dose | Recovery |
|---|---|---|---|
| Control | 206 ± 13 | 202 ± 4 | 201 ± 7 |
| 5% Blood | 184 ± 15 | 189 ± 14 | 182 ± 8 |
| Drawdown | | | |
| REGN5381 | 188 ± 8 | 169 ± 5*** | 171 ± 7* |

±SEM;
*p < .05,
***p < .001 vs. controls

REGN5381 significantly reduced mean plasma volume and was effective at reducing plasma volume as a 5% blood drawdown.

### Example 3. Acute Hemodynamic Assessment of REGN5381 in Anesthetized Beagle Dogs

### Experimental Procedure

The purpose of this study was to evaluate the acute hemodynamic profile of REGN5381 when administered to anesthetized male beagle dogs via a single IV bolus injection. In order to evaluate the acute hemodynamic profile of REGN5381 when administered to anesthetized male beagle dogs via a single IV bolus injection, prior to dose administration, each animal was anesthetized and instrumented for acquisition of cardiovascular hemodynamic data and urine collection. Animals each received a single IV bolus dose of saline (n=6), 10 mg/kg valsartan (n=4), or 25 mg/kg REGN5381 (n=6). Following dose administration, animals were monitored for 4 hours to assess hemodynamic changes. The left-ventricular and systemic pressure waveforms were collected for each animal pre-dose and during the 4-hour post-dose monitoring period; pre-dose measurements served as the baseline for each animal. The end systolic volume and end diastolic volume were assessed using transthoracic echocardiogram pre-dose and post-dose once hourly. Urine was collected from the bladder, and its volume was measured pre-dose and post-dose once hourly. Perfusion of the renal cortex and medulla, measured using neutron-activated microspheres, was determined pre-dose and 2 and 4 hours post-dose for some animals receiving REGN5381 (n=4) and saline (n=4). Pre-dose measurements served as the baseline for each animal.

### Results

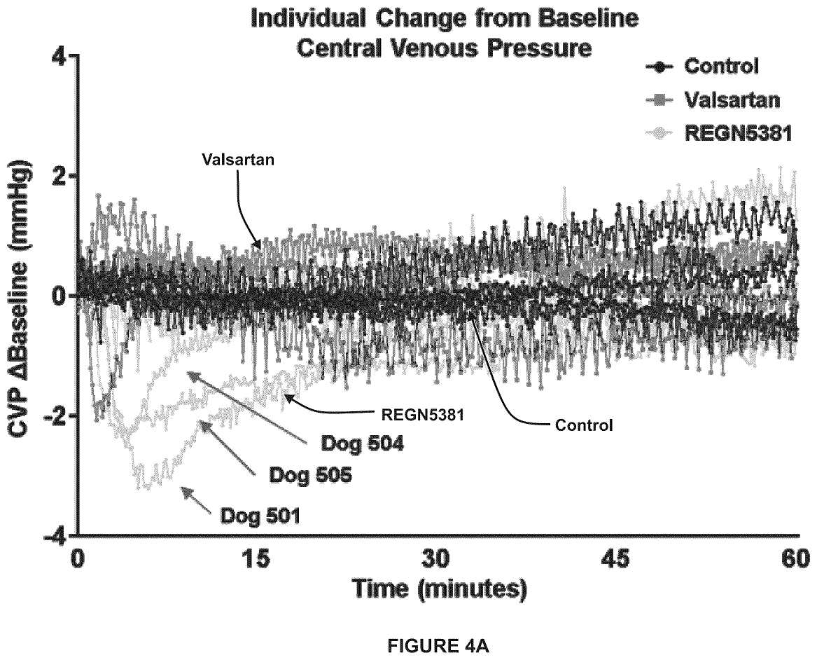

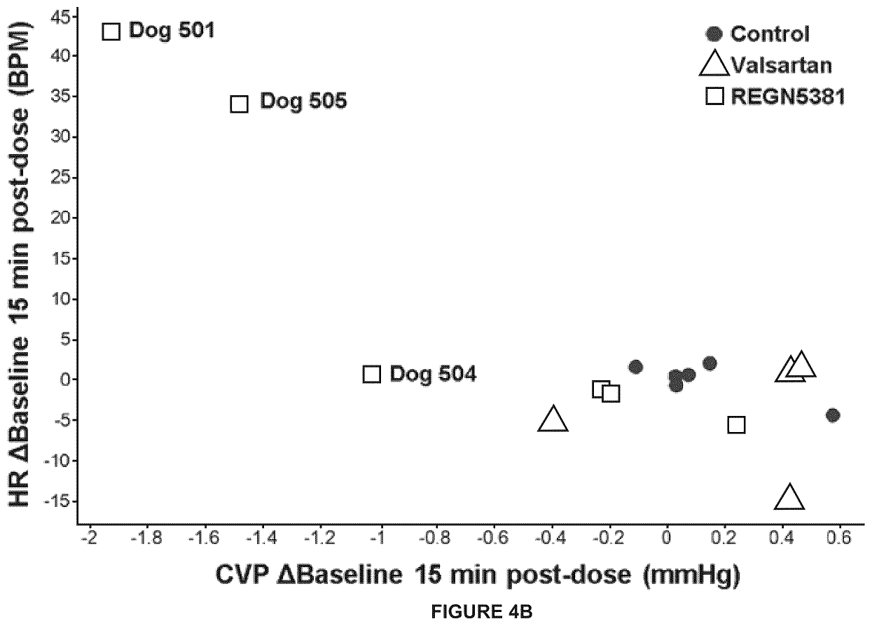

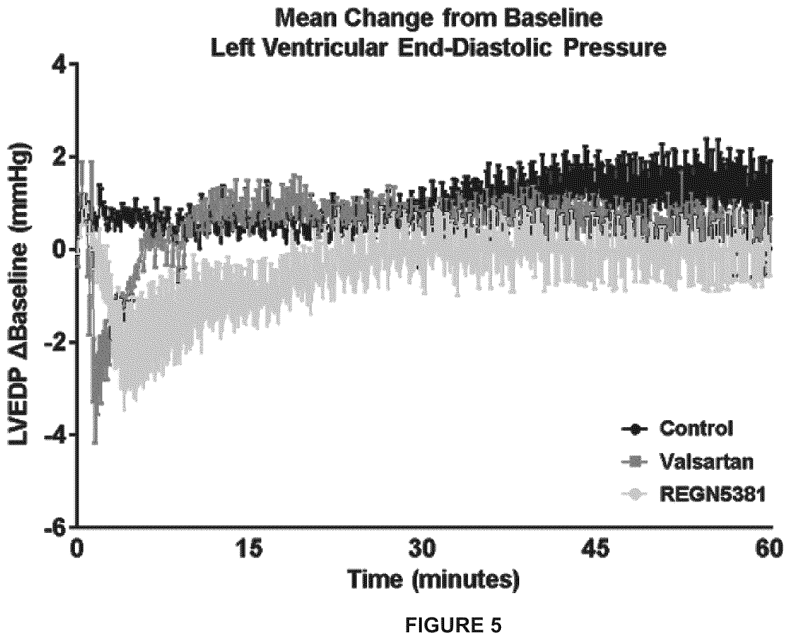

REGN5381 resulted in an acute transient mean reduction from baseline central venous pressure (CVP) accompanied by an acute transient mean elevation from baseline HR during the first hour post-dose. The observed changes in mean CVP and HR were attributed to readings from 3 of 6 animals that received REGN5381, where the degree of change in CVP correlated with the degree of change in HR (FIGS. 4A and 4B). Furthermore, a mean reduction from baseline left ventricular end-diastolic pressure (LVEDP) during the first hour post-dose was observed and attributed to the same 3 animals, consistent with REGN5381-induced pre-load reduction consistent with the observed effects on CVP in these animals (FIG. 5).

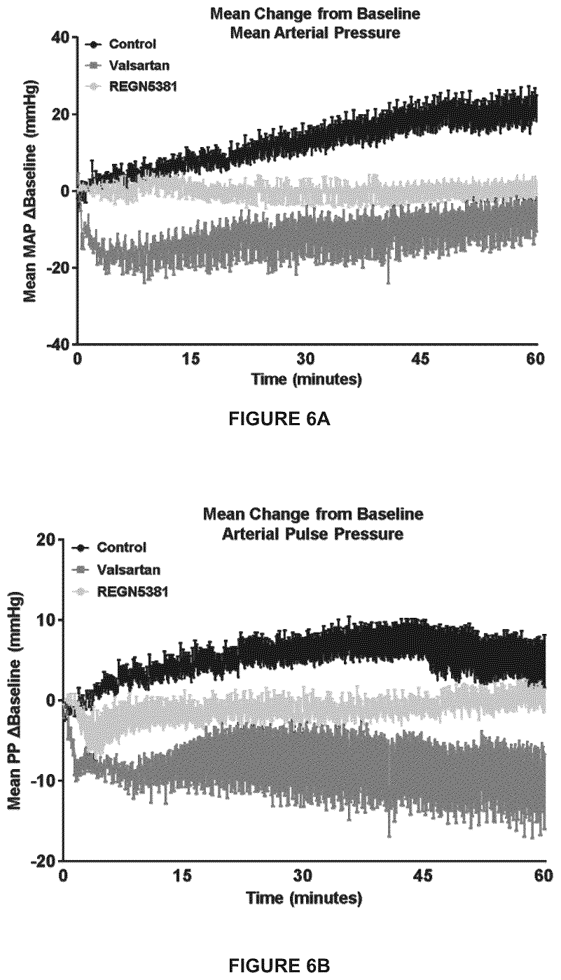

In the other 3 animals that received REGN5381, the post-dose CVP and heart rate during the first hour post-dose were similar to baseline levels. Exposure assessment indicated that all animals had comparable REGN5381 serum concentrations (data not shown). Consistent with the arterial systolic pressure data, no change in MAP or systemic vascular resistance (SVR) were observed. A transient mean reduction from baseline of arterial pulse pressure (PP) was observed (FIGS. 6A and 6B). The lack of changes in arterial pressure and the transient nature of any hemodynamic effects were not consistent with that observed in conscious telemetered dogs (data not reported), non-human primates (data not shown), and mice (data not reported, likely due to experimental conditions associated with this surgical procedure, including but not limited to anesthesia (both induction and maintenance), lateral recumbent positioning during data acquisition, and mechanical ventilation.

No mean changes in CVP or HR were observed in animals that received valsartan during the first hour post-dose; an early, transient mean reduction from baseline LVEDP was observed in animals that received valsartan during the first-hour post-dose; however, this was attributed to a reading from 1 animal (likely due to poor catheter placement). A mean reduction from baseline MAP and PP during the first hour post-dose, as well as a mean reduction in SVR throughout the 4-hour monitoring period, were observed in animals that received valsartan, consistent with the mechanism of action of valsartan as an angiotensin Il receptor blocker.

No mean changes in CVP, HR, LVEDP, PP, or SVR were observed in animals that received saline. A slight increase in MAP was observed in animals that received saline; however, this effect was likely due to surgical preparation (i.e., temperature and/or depth of anesthesia plane).

Collectively, in the dogs that responded to REGN5381, these results indicate that REGN5381 induces robust transient effects on venous pressure with a correlative reflex tachycardic response when administered to anesthetized, laterally recumbent positioned, instrumented normotensive male beagle dogs. The observed hemodynamic changes are independent of any change in urine output or systemic organ perfusion. The observed REGN5381-induced hemodynamic effects are different than induced by the standard of care, valsartan, suggesting a different mechanism of action for the REGN5381-induced hemodynamic effects.

Finally, REGN5381 did not affect renal perfusion or induce diuresis. No changes from baseline perfusion of the renal cortex or renal medulla at 2 and 4 hours post-dose were observed in animals that received REGN5381. In addition, no change from baseline urine output was observed among animals that received REGN5381 at the timepoints assessed. When comparted to time-matched control animals, a significant increase of urine output was observed at 1 and 2 hours post-dose among animals that received valsartan. No mean changes in urine output or renal perfusion were observed in animals that received saline.

Thus, in a study assessing the acute hemodynamics of a single dose of 25 mg/kg REGN5381 administered IV in anesthetized dogs, REGN5381 acutely reduced venous pressures in 3 of 6 animals during the first hour post-dose with no effect on arterial pressures. REGN5381 induced an acute mean reduction of central venous pressure (CVP) compared to both baseline and time-matched control animal values. In concert with the observed acute reduction of CVP, animals dosed with REGN5381 experienced an acute, transient increase of heart rate (HR) when compared to both baseline and time-matched control animals during the first hour post-dose. Evaluation of individual animal data indicated that the hemodynamic and associated HR changes were limited to 3 of the 6 animals that received REGN5381, where the magnitude of CVP reduction correlated with the degree of change in HR. Arterial pressures were not affected by REGN5381 in this anesthetized model, as both change from baseline and comparison to time-matched control mean arterial pressures (MAP) were not different. REGN5381-induced hemodynamic changes did not affect organ perfusion as no changes from baseline or compared to time-matched controls were noted for perfusion of the renal cortex or renal medulla at 2 and 4 hours post-dose. In addition, no REGN5381-related effect on urine output was observed when assessed hourly post-dose. The observed variation of response to REGN5381 could be related to interindividual differences in NPR1 expression and/or function, attributed to the anesthetized preparation and anesthetic plane of each animal, as well as the mechanical ventilation and lateral recumbent positioning of the dogs. The observed magnitude of reduction in venous pressure correlated with the associated degree of increase in heart rate during the first hour post-dose. This effect is likely a compensatory reflex tachycardic response to maintain cardiac output in the REGN5381-responding animals.

### Example 4. A Single Subcutaneous and Intravenous Injection Pharmacokinetic and Pharmacodynamic Study Assessing REGN5381 in Telemetered Male Cynomolgus Monkeys

The objective of this study was to determine the magnitude and duration of effect of a single intravenous (IV) bolus or subcutaneous (SC) injection of REGN5381 on systemic blood pressure in telemetered male cynomolgus monkeys. In addition, the pharmacokinetics (PK) of REGN5381 was assessed.

Thirty male cynomolgus monkeys were assigned to one of 6 groups (5 animals/group) (Table 9). Animals received a single SC injection of control article or 1, 5, or 25 mg/kg REGN5381, or a single IV bolus injection of 5 or 25 mg/kg REGN5381.

TABLE 9
Study design for the single-dose pharmacokinetic and pharmacodynamic
study of REGN5381 administered via subcutaneous or intravenous
injection to male cynomolgus monkeys

| | | | Dose | | |
|---|---|---|---|---|---|
| | | | Level | Dose | Number of |
| | | Dose | (mg/kg/ | Concentration | Animals |
| Group | Test Material | Route | day) | (mg/mL) | (Male) |
| 1 | Control (saline) | SC | 0 | 0 | 5 |
| 2 | REGN5381 | SC | 1 | 1 | 5 |
| 3 | REGN5381 | SC | 5 | 5 | 5 |
| 4 | REGN5381 | SC | 25 | 25 | 5 |
| 5 | REGN5381 | IV | 5 | 5 | 5 |
| 6 | REGN5381 | IV | 25 | 25 | 5 |

IV, Intravenous;
SC, Subcutaneous
Note:
REGN5381 was administered via a subcutaneous injection or an intravenous slow bolus injection at a volume of 1 mL/kg.

Notes: CL/F was calculated for SC groups, CL was calculated for IV groups.

Bioavailability was calculated using AUClast from the SC group and comparing it with the IV dose group at the same dose level.

Blood samples for the determination of total REGN5381 concentrations in serum were collected from all animals prior to dosing through Day 56.

Blood samples, processed to plasma or serum, were collected from all animals prior to dosing and at selected timepoints for the analysis of biomarkers including cyclic guanosine monophosphate (cGMP) and/or N-terminal proatrial natriuretic peptide (NTproANP). Urinalysis for cGMP was also conducted at these time points.

Electrocardiography (ECG) and blood pressure were recorded once during pretreatment (Week-1) for a 24-hour period and over 4 weeks post dose (3 hours prior to dosing through Day 28), then once weekly for 24 hours from Day 29 through Day 56.

### Results

Following REGN5381 administration at dose levels ranging from 1 to 25 mg/kg, concentration-time profiles of total REGN5381 are characterized by an initial brief distribution phase (IV) or absorption phase (SC), followed by a linear beta elimination phase at high concentrations, consistent with saturation of the target-mediated clearance (TMC) pathway, and a nonlinear elimination phase at low concentrations reflective of TMC. The linear beta phase is primarily observed at the 5 and 25 mg/kg dose levels.

Following an IV dose of REGN5381 at 5 and 25 mg/kg, Cₘₐₓ increased in a dose-proportional manner, as indicated by nearly equivalent dose-normalized Cₘₐₓ values.

Following IV administration, a deviation from dose proportional increase in exposure (AUCₗₐₛₜ) was observed across the two dose levels, indicative a tread towards nonlinear kinetics. Total body clearance (CL) decreased slightly with increasing dose and the associated increasing concentrations, from 4.80 mL/day/kg in the 5 mg/kg group to 3.95 mL/day/kg in the 25 mg/kg group.

Following a SC dose of REGN5381 at 1, 5, and 25 mg/kg, a dose-proportional increase in Cₘₐₓ was observed. Mean tₘₐₓ was observed in the range of 1.41 to 1.81 days. In the three SC dose groups, a greater than dose proportional increase in exposure (AUCₗₐₛₜ) was observed. The calculated bioavailability following 5 and 25 mg/kg SC was essentially complete.

Elimination half-life (t_{1/2}) was estimated in the terminal phase of concentration-time profiles. The estimates were generally comparable between IV and SC doses at the same dose level. In the 25 mg/kg groups where the linear beta phase was observed throughout the study duration, a half-life of approximately 9 to 11 days was estimated.

TABLE 10
Mean Pharmacokinetic Parameters of Total REGN5381 in Serum Following a Single
Subcutaneous or Intravenous Injection of REGN5381 in the Male Cynomolgus Monkey

| | | REGN5381 | | | REGN5381 | | | REGN5381 | | | REGN5381 | | | REGN5381 | | |
|---|---|---|---|---|---|---|---|---|---|---|---|---|---|---|---|---|
| | | 1 mg/kg SC | | | 5 mg/kg SC | | | 25 mg/kg SC | | | 5 mg/kg IV | | | 25 mg/kg IV | | |
| Parameter | Unit | N | Mean | SD | N | Mean | SD | N | Meat | SD | N | Mean | SD | N | Mean | SD |
| Cₘₐₓ | μg/mL | 5 | 13.6 | 3.84 | 5 | 70.1 | 13.0 | 5 | 358 | 56.0 | 5 | 83.9 | 7.69 | 5 | 425 | 30.0 |
| Cₘₐₓ/Dose | (μg/mL)/ | 5 | 13.6 | 1.84 | 5 | 14.0 | 2.61 | 5 | 14.3 | 2.24 | 5 | 16.8 | 1.54 | 5 | 17.0 | 1.20 |
| | (mg/kg) | | | | | | | | | | | | | | | |
| tₘₐₓ | day | 5 | 1.81 | 0.842 | 5 | 1.61 | 0.548 | 5 | 1.41 | 0.547 | 5 | 1.00 | 0.0138 | 5 | 1.00 | 0.0130 |
| AUCₗₐₛₜ | day · | 4 | 108 | 16.0 | 5 | 1090 | 90.9 | 5 | 6110 | 1290 | 3 | 1050 | 72.1 | 3 | 6220 | 410 |
| | (μg/mL) | | | | | | | | | | | | | | | |
| AUCₗₐₛₜ/ | day · | 4 | 108 | 16.0 | 5 | 219 | 18.2 | 5 | 244 | 51.6 | 3 | 210 | 14.4 | 3 | 249 | 16.4 |
| Dose | (μg/mL)/ | | | | | | | | | | | | | | | |
| | (mg/kg) | | | | | | | | | | | | | | | |
| CL/F (CL) | mL/day/kg | 4 | 7.44 | 1.57 | 5 | 4.57 | 0.408 | 5 | 4.15 | 1.25 | 3 | 4.80 | 0.330 | 3 | 3.95 | 0.350 |
| terminal t_{1/2} | day | 4 | 5.66 | 0.959 | 5 | 6.34 | 1.46 | 5 | 11.2 | 2.87 | 3 | 4.12 | 0.107 | 3 | 9.20 | 3.05 |
| Vₛₛ | mL/kg | 0 | NC | NC | 0 | NC | NC | 0 | NC | NC | 3 | 49.7 | 3.84 | 3 | 56.0 | 7.62 |

| | | | | | |
|---|---|---|---|---|---|
| Bioavailability | — | 104 | 98.2 | — | — |

* AUC, Area under the concentration-time curve; AUClast, AUC computed from time zero to the time of the last detectable concentration; Cmax, Peak concentration; CL, Total body clearance; CL/F, Apparent total body clearance; IV, Intravenous; N, Number of animals; NC, Not calculated; SC, Subcutaneous; SD, Standard deviation; tmax, Time to Cmax; terminal t1/2, terminal elimination half-life; Vss, Volume of distribution at steady state; —, Not applicable

There was an REGN5381-related decrease in blood pressure in all dose groups. The overall magnitude of change was similar at the three dose levels; however, a sustained hypotensive effect was observed for the entirety of the recording session (56 days) with the 25 mg/kg SC and IV dose groups, while mean blood pressure trended to values comparable to the control group by end of the recording session (56 days) for the 1 and 5 mg/kg dose groups.

Of note, REGN5381 induced significant and persistent reductions of systemic blood pressure, with no evidence of adverse hypotension (i.e., syncope, altered locomotion, death) in telemetered male cynomolgus monkeys.

### Example 5. A Randomized, Double-blind, Placebo-controlled, Two-part Single Ascending Dose Study to Assess the Safety, Tolerability, and Pharmacokinetics of REGN5381 in Humans

### Study Objectives

The primary objective of the instant study is to evaluate the safety and tolerability of single intravenous (IV) doses of REGN5381 in healthy normotensive and otherwise healthy hypertensive adults.

The secondary objectives of the instant study are to evaluate the effect of single IV doses of REGN5381 on blood pressure (BP) and heart rate (HR) in healthy normotensive and otherwise healthy hypertensive adults; to evaluate the effect of single IV doses of REGN5381 on cardiac stroke volume (SV); to evaluate the pharmacokinetics (PK) of single IV doses of REGN5381; and to evaluate the immunogenicity of single IV doses of REGN5381.

The exploratory objectives of the instant study are to evaluate the effect of single IV doses of REGN5381 on serum and urinary pharmacodynamic (PD) biomarkers of natriuretic peptide receptor 1 (NPR1) agonism indicative of target engagement; to evaluate the effect of single IV doses of REGN5381 on additional exploratory biomarkers related to myocardial and renal function; to evaluate the effect of single IV doses of REGN5381 on cardiac contractility, respiratory variation of cardiac performance, and systemic vascular resistance (SVR); to evaluate the effect of single IV doses of REGN5381 on diuresis and natriuresis; to evaluate the effect of single IV doses of REGN5381 on BP variability; to evaluate the hemodynamics effects of a crystalloid fluid bolus in subjects who receive single IV doses of REGN5381; and to conduct exploratory research related to the safety and efficacy of REGN5381, NPR1, and BP.

### Study Endpoints

The primary endpoint in the instant study is the type, incidence, and severity of treatment-emergent adverse events (TEAEs) following single IV dose administrations of REGN5381 or placebo over time.

The secondary endpoints in the instant study are: change from baseline in systolic blood pressure (SBP), diastolic blood pressure (DBP), mean arterial pressure (MAP), pulse pressure (PP), heart rate (HR), and stroke volume (SV) over time; maximum change from baseline in SBP, DBP, MAP, PP, HR, and SV across the first 24 hours post-dose; change from baseline in the 24-hour mean SBP, DBP, MAP, PP, and HR measured from 0 to 24 hours, 24 to 48 hours, and 48 to 72 hours post-dose; concentrations of REGN5381 over time; and number and percentage of subjects who develop anti-drug antibodies (ADA) and titers over time.

The exploratory endpoints of the instant study are: change from baseline in urine cGMP and plasma cGMP over time; change from baseline in renin, aldosterone, N-terminal (NT)-proBNP and cardiac troponin T after dose administration over time; change from baseline in derivative of blood pressure over time (dP/dt); change from baseline in urine output and sodium clearance over time; change from baseline in BP variability over time; and change in SV, stroke volume variation (SVV), SBP, DBP, and MAP over time following a crystalloid fluid bolus.

### Study Variables

Study variables include: i) Demographic and Baseline Characteristics (baseline

characteristics will include standard demography (e.g., age, race, weight, height, etc.), medical history, and medication history for each subject); ii) Safety Variables (the safety variables include oscillometric BP and HR (both semi-recumbent and orthostatic), telemetry, other vital signs (e.g., temperature and respiratory rate), physical examination, electrocardiograms (ECGs), laboratory evaluations (hematology, chemistry, and urinalysis), and adverse events (AEs)); iii) Pharmacokinetic variables (the PK variable is the concentration of total REGN5381 and time); iv) Immunogenicity variables (the immunogenicity variables are ADA status and titer, and nominal sampling time/visit); and v) Pharmacodynamic and other Biomarker variables (pharmacodynamic variables are derived from the continuous pulse wave analysis (PWA) and include SBP, DBP, MAP, cardiac output, SV, SVV, SVR, and dP/dT; additional biomarker variables to be assessed include 24-hour urine cGMP, plasma cGMP, plasma renin, plasma aldosterone, plasma N-terminal-(NT)-proBNP, and plasma high sensitivity cardiac troponin-T (hs-cTnT); the biomarker variables are the concentrations of each biomarker in urine (24-hour urine cGMP) or plasma (cGMP, renin, aldosterone, NT-proBNP and plasma hs-cTnT) at each time point that a sample is collected).

### Study Design:

The instant study is a 2-part, phase 1, randomized, double-blind, placebo-controlled first-in-human (FIH) study of REGN5381. The objective of this study is to test the safety, tolerability, PK, and PD of single ascending IV doses of REGN5381, an agonist mAb to NPR1, in healthy normotensive and otherwise healthy hypertensive adults between 18 and 55 years of age. The study will be performed in 2 sequential parts (Part A and Part B). Part A constitutes a single ascending dose (SAD) study of REGN5381 in healthy normotensive and otherwise healthy hypertensive adults to assess safety and tolerability over a series of doses. Part B consists of an assessment of the hemodynamic response of IV fluid bolus after a selected dose of REGN5381.

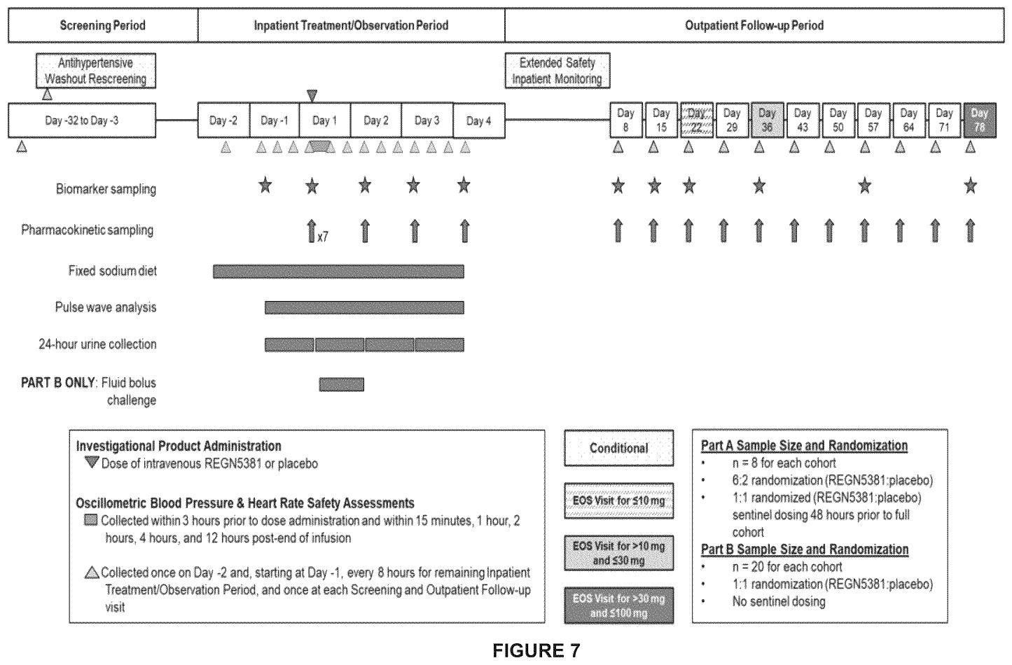

Parts A and B of the study both comprise a screening period (day −32 to day −3), a conditional antihypertensive washout rescreening period (day −25 to day −3, described below), an inpatient treatment/observation period (day −2 to day 4), a conditional extended safety inpatient monitoring period (day 5 to day 21), and an outpatient follow-up period (day 8/week 2 to day 78/week 12). On study day 1, subjects will be randomized to receive REGN5381 (stored at the site at a temperature of 2° C. to 8° C.) or placebo in a 6:2 ratio. Study drug will be administered as an IV infusion (FIG. 7).

Subjects who present at the initial screening on a stable antihypertensive regimen

consisting of a single antihypertensive agent may be eligible for the study but must undergo a washout period of at least 1 week or 5 half-lives, whichever is longer, but not to exceed a maximum of 8 weeks. Subjects are not eligible for washout if they are on more than 1 antihypertensive agent or have an SBP exceeding 140 mm Hg during the screening visit. Subjects undergoing antihypertensive washout will be asked to report any new symptoms, including home BP assessments, if available. Subjects may be asked to restart their antihypertensive treatment, and will not be considered eligible for the study. After these subjects have been appropriately washed off the antihypertensive agent, they must return for a washout rescreening visit to confirm eligibility for the study prior to randomization. Subjects will hold their antihypertensive agent until the end of the inpatient treatment/observation period. Subjects may resume their home antihypertensive agent any time after inpatient discharge.

Study eligibility will be determined using BP and HR measurements at screening:
Part A: systolic blood pressure (SBP)≥100 mm Hg and ≤140 mm Hg and diastolic blood pressure (DBP)≥60 mm Hg and ≤90 mm Hg for early cohort(s), with an option to adapt enrollment to subjects with an SBP≥130 mm Hg and ≤165 mm Hg and DBP≥60 mm Hg and ≤100 mm Hg (described below); and
Part B: SBP≥130 mm Hg and ≤165 mm Hg and DBP≥60 mm Hg and ≤100 mm Hg.

With respect to adapting enrollment, the BP inclusion criteria for Part A may be adapted (decision will be made based on a review of aggregated safety data including BP and HR from early cohort(s) according to the following criteria:
Three or more subjects have a decrease in SBP or DBP exceeding 15 mm Hg below baseline from day 1 (predose) that is sustained for more than 2 hours;
Three or more subjects have both an HR>110 bpm and an increase in HR of at least 20 bpm above baseline from day 1 (predose) that is sustained for more than 2 hours; or.
Two or more subjects require rescue therapy.
   If criteria to adapt enrollment are met, the dose level will be decreased, or if already at the lowest dose, repeated.

Notable exclusion criteria for this study include a history of cardiovascular disease (including stroke/transient ischemic attack [TIA]), risk factors for cardiovascular disease including diabetes, hyperlipidemia, a history of severe hypertension (SBP>180, or DBP>110 mm Hg), and history of unexplained syncope, autonomic dysfunction, or neurologic disease.

Eligible subjects who sign informed consent will be admitted to an inpatient

treatment/observation unit on study day −2. During the inpatient stay, subjects will be monitored using a combination of automated oscillatory BP recordings, continuous PWA, and cardiac telemetry. Site preparations to treat any signs or symptoms related to expected or unanticipated pharmacology will include the following: availability of trained staff, 2 IV catheters including one≤18 gauge placed prior to drug infusion and left for a minimum of 4 hours, and readily available rescue therapies.

Detailed hemodynamic assessments will occur during the inpatient treatment/observation period. Subjects will be provided a fixed sodium diet (approximately 3,000 mg daily) for the length of the inpatient treatment/observation period, and total volume of fluid intake and urine output will be recorded. Orthostatic BP will be collected on day −1 to ensure optimal fluid status prior to receiving REGN5381. Total oral fluid intake on day −1 and day 1 will be kept within a fixed range for all subjects.

The length of the inpatient treatment/observation period following study drug administration will be at least 72 hours. Strict discharge and study stopping criteria are provided to ensure adequate recovery of hemodynamic effects before discharge. Following discharge, subjects will attend a series of weekly outpatient follow-up assessments to monitor safety and PK up to a maximum of 11 weeks. These assessments may be completed during extended inpatient safety monitoring if additional monitoring is required. There are no dietary restrictions during extended inpatient safety monitoring or outpatient period.

For Part B only, a fluid bolus will be administered in otherwise healthy hypertensive adults. Further descriptions of cohorts and dose escalation are provided below. Part B dose selection will be chosen based on the available PD and safety results through day 8 from the highest dose cohort in Part A.

Subjects in cohorts 10 and 11 will receive a 500 to 1,000 cc crystalloid fluid bolus 2 to 24 hours after study drug administration to assess hemodynamic response. The exact volume and timing of the crystalloid fluid bolus, which will be the same across the cohort, will be determined.

The anticipated total duration of this FIH study is approximately 65 weeks, including Part A and Part B.

### Study Drug

REGN5381 drug product is supplied as a 12.5 mg lyophilized powder in a sterile single-use vial. Subjects in Part A and Part B will receive either REGN5381 IV or matching placebo IV as a single dose administered via IV infusion at a rate of 20 mL/hr using a syringe pump. Anticipated dose levels for Part A will include 0.3 mg IV up to a planned maximal dose of 100 mg IV. Dose level(s) in Part B will not exceed the doses tested in Part A and will be based on a review of Part A data. Placebo to match REGN5381, is supplied as a lyophilized powder in a sterile single-use vial.

### Study Cohorts, Part A

Up to 72 normotensive or otherwise healthy hypertensive subjects will be enrolled and randomized in up to 9 sequential ascending dose cohorts, to include doses of 0.3 mg up to 100 mg. Cohorts 8 and 9 are optional cohorts that may not exceed a dose of 100 mg. These additional cohorts may be used to better understand the safety, tolerability, pharmacokinetic (PK), and pharmacodynamic (PD) of REGN5381 based on accumulated PD and safety data from Cohorts 1 to 7.

Each dose cohort will consist of 8 subjects: 6 randomized to receive a single IV dose of REGN5381 and 2 randomized to receive placebo. To optimize safety, the first 2 subjects in each cohort (1 active: 1 placebo) will be enrolled as a sentinel group and will be dosed at least 48 hours prior to the remaining subjects. The remaining subjects will only be dosed after both subjects in the sentinel cohort have safely completed at least 48 hours of safety assessments, the safety data have been reviewed, and a decision to continue study drug dosing has been documented. Sentinel subjects are not required if repeating or going down a dose level.

A 3 to 3.3-fold dose escalation between cohorts is planned, according to the dose escalation criteria below. Dose escalations may be adjusted based upon the observed PD response within each cohort; between-dose level escalations of smaller than 3 to 3.3-fold increments may be warranted. The maximum dose will not exceed 100 mg.

Throughout the study, an ongoing review of the available PD and safety data will be used to inform duration of assessments and dose escalation for the remaining cohorts. Cohort 5 will repeat a ≤10 mg dose in subjects with mild hypertension. Dose selection for Cohort 8 will be made after the complete PD and safety data through Day 8 have been reviewed from Cohorts 1 to 7. Data from cohort 8 through day 8 will inform dose selection for Cohort 9.

Ascending dose cohorts will be enrolled as follows:
Cohort 1: REGN5381 at 0.3 mg IV (or placebo), single dose
Cohort 2: REGN5381≤1 mg IV (or placebo), single dose
Cohort 3: REGN5381≤3 mg IV (or placebo), single dose
Cohort 4: REGN5381≤10 mg IV (or placebo), single dose
Cohort 5: REGN5381≤10 mg IV (or placebo), single dose
Cohort 6: REGN5381≤30 mg IV (or placebo), single dose
Cohort 7: REGN5381≤100 mg IV (or placebo), single dose
Cohort 8 (Optional): REGN5381≤100 mg IV (or placebo), single dose
Cohort 9 (Optional): REGN5381≤100 mg IV (or placebo), single dose

### Study Cohorts, Part B

Up to 40 hypertensive but otherwise healthy subjects will be enrolled in up to 11 cohorts (cohort 11 is optional). In each cohort, 10 subjects will be randomized to receive a single IV dose of REGN5381 and 10 randomized to receive placebo. These two cohorts in Part B will not require a sentinel group.

Dose level(s) in Part B will not exceed the doses tested in Part A and will be based on review of the available safety and PD data up to day 8 in the highest dose cohort. The optional cohort may be used to evaluate an additional dose level from the range previously evaluated in Parts A and B. The inclusion of a final, optional cohort in Part B will be based on available data from Parts A and B, including the available safety and PD data. Dose cohorts will be enrolled as follows:
Cohort 10: REGN5381≤100 mg IV, single dose (or placebo)+IV fluid bolus
Cohort 11 (Optional): REGN5381≤100 mg IV, single dose (or placebo)+IV fluid bolus
   The objective of Part B is to characterize the hemodynamic response to IV fluids after selected doses of REGN5381.

Parts A and B of the study both comprise a screening period, a conditional antihypertensive washout rescreening period, an inpatient treatment/observation period, a conditional extended safety inpatient monitoring period, and an outpatient follow-up period.

### Dose Escalation, Part A

Enrollment will begin into dose cohort 1. The decision to escalate to the next higher dose cohort will be made after review of safety data through day 8. Aggregated safety data will be reviewed to determine the dose level to be tested in the next cohort, including possible dose escalation, dose level lowering, repeating of a dose level, adapting enrollment to a higher BP range, or stopping study drug dosing altogether and proceeding to Part B.

As part of the aggregate safety data review, dose escalation will not occur if 3 or more subjects receiving REGN5381 require extended monitoring for hemodynamic effects beyond 72 hours, according to the individual subject discharge criteria described below.

### Individual Subject Discharge Criteria, Parts A and B

At 72-hours post-dose, the subject will undergo oscillometric BP and HR assessment and must meet all the following criteria to be eligible for discharge from inpatient monitoring:
Median SBP≥90 mm Hg; and.
Median HR≤100 bpm or HR less than or equal to 1.2×(≤120%) of baseline from day 1 (predose), whichever is higher;
No unresolved AEs that warrant continued inpatient monitoring (Note: If the subject has unresolved AEs at 72-hours post-dose, the subject may be discharged, if the first 2 criteria are met and the AEs may be appropriately managed in an outpatient setting).

If 3 or more subjects receiving REGN5381 require extended monitoring, one may opt to repeat a dose cohort, decrease the dose level, and/or adapt enrollment for the remainder of Part A. Alternatively, Part A dose escalation may be ended, and the study may proceed to Part B. A 3 to 3.3-fold dose escalation interval between cohorts is planned, however this interval may be reduced. If the decision is made to adapt enrollment to subjects with mild hypertension, the dose level will be decreased, or if already at the lowest dose, repeated using adapted enrollment criteria.

Subjects who do not meet the individual subject discharge criteria at 72 hours post-dose will be kept for extended safety inpatient monitoring. The subjects will be assessed every additional day until they are able to meet the discharge criteria. Modifications to the dose, dosing regimen, and/or clinical or laboratory procedures (including timing of PK and biomarker sampling) currently outlined may be required to achieve the scientific goals of the study objectives and/or to ensure appropriate safety monitoring of the study participants. As such, some alterations from the currently outlined dose and/or dosing regimen may be permitted based on newly available data, but the maximum dose may not exceed those currently outlined in the protocol. Changes permitted within protocol parameters include possible dose escalation, dose level lowering, repeating of a dose level, adapting enrollment to a higher BP range (as described above), or stopping study drug dosing altogether and proceeding to Part B. Entire cohorts may also be omitted, and, if necessary, a pause between cohorts may be requested for review of the available PK, PD, and safety data.

Some subjects may not meet discharge criteria at 72 hours due to a reduction of BP secondary to inactivity or low-sodium diet during inpatient treatment/observation; therefore, decisions regarding dose escalation will be made based on subjects who receive REGN5381 and complete day 8 safety assessments. The flexibility to repeat a dose level or decrease to a lower dose allows for additional characterization of the extent and duration of hemodynamic effects and other safety parameters.

The end of study (EOS) is defined as the date the last subject completes the last study visit, withdraws from the study, or is lost to follow-up (i.e., the study subject can no longer be contacted).

### Study Subjects

Up to 112 adults (up to 72 in Part A, and 40 in Part B) will be enrolled, randomized, and dosed in this study. Given that the dose escalation stopping criteria in Part A is based on responses to REGN5381 (safety data including, but not limited to, BP and HR) and the optional cohorts in Parts A and B, it is possible that fewer subjects will be needed to meet study objectives. Eligible subjects for this study include healthy males and females, 18 to 55 years of age (inclusive), with normal or mildly elevated BP.

Inclusion criteria for subjects:
Male or female subjects 18 to 55 years of age (inclusive) at the initial screening;
SBP and DBP must be within 20 mm Hg between measurements in both of the subjects' arms at the initial screening visit;
For Part A, SBP between 100 and 140 mm Hg (inclusive) and DBP between 60 and 90 mm Hg (inclusive) at the initial screening, repeatable once during the screening period (Note: the BP inclusion criteria for Part A may be adapted based on a review of aggregated safety data to an SBP between 130 and 165 mm Hg (inclusive) and DBP between 60 and 100 mm Hg (inclusive) at the initial screening, repeatable once during the screening period);
For Part B, SBP between 130 and 165 mm Hg (inclusive) and DBP between 60 and 100 mm Hg (inclusive) at the initial screening, repeatable once during the screening period;
SBP of at least 100 mm Hg on study day 1, prior to study drug administration;
Resting HR between 45 and 100 bpm (inclusive) at the initial screening, repeatable once during the screening period;
Body mass index between 18 and 33 kg/m² (inclusive) at the initial screening;
Subject is judged to be in good health based on medical history, physical examination, vital sign measurements, and ECG performed at screening and/or prior to study drug administration;
Subject is in good health based on laboratory safety testing obtained during the screening period (Note: any clinically insignificant abnormal laboratory results (e.g., creatine phosphokinase within 3× upper limit of normal; with suspected cause due to rigorous physical activities), may be repeated once during the screening period; if the repeat test is within normal range or not clinically significant outside of normal range, the subject may be enrolled);
Pregnancy tests must be negative prior to study drug administration;
Willing and able to comply with clinic visits and study-related procedures; and
Provide informed consent signed by study subject.

### Exclusion Criteria for Subjects:

Active hypertension requiring treatment with 2 or more antihypertensive agents; history of severe hypertension (SBP>180, or DBP>110 mm Hg); a) Subjects on single drug therapy for hypertension may enroll after at least a 1-week (or at least 5 half-lives, whichever is longer) washout period; subjects are not eligible for washout if they are on treatment with more than 1 antihypertensive agent, high-dose beta-blocker therapy, or have an SBP exceeding 140 mm Hg during the screening visit; b) Subjects may wash out of single-agent low-dose beta-blocker therapy, defined as ≤100 mg metoprolol total daily dose, ≤25 mg carvedilol total daily dose, ≤50 mg atenolol total daily dose, ≤80 mg propranolol total daily dose, ≤5 mg bisoprolol total daily dose or equivalent doses of other beta-blockers; c) The washout period may not extend beyond 8 weeks prior to dose administration; subjects may resume antihypertensive therapy at the time of inpatient discharge;
History of Raynaud's, any prior finger injury that would preclude wearing a non-invasive PWA device, or inadequate arterial pulses in the bilateral digits as determined by the PWA signal;
History of unexplained syncope or autonomic dysfunction;
History of clinically significant cardiovascular (including stroke/TIA), respiratory, hepatic, renal, gastrointestinal, endocrine, hematological, psychiatric or neurological disease that may confound the results of the study or pose an additional risk to the subject by study participation;
Any physical examination findings and/or history of any illness that might confound the results of the study or pose an additional risk to the subject by study participation;
Hospitalization (>24 hours) for any reason within 30 days of the screening visit;
Current or former nicotine use (combustible cigarettes or electronic nicotine delivery system, including e-cigarettes) who stopped smoking within 3 months prior to screening, or current user of nicotine replacement therapy; cotinine test must be negative at screening and prior to study drug administration;
History of drug or alcohol abuse within a year prior to the screening visit; drug and alcohol tests must be negative at screening and prior to study drug administration;
Presence of human immunodeficiency virus (HIV), hepatitis B virus (HBV), or hepatitis C virus (HCV) seropositivity at screening or within 3 months prior to study drug administration with the exception of false positive screening tests as documented by polymerase chain reaction or Western blot; HCV-seropositive subjects with documentation of 12 months sustained virologic response may still be enrolled;
Any malignancy within the past 5 years, except for basal cell or squamous epithelial cell carcinomas of the skin or carcinoma in situ of the cervix or anus, that have been resected, with no evidence of metastatic disease for 3 years;
Estimated glomerular filtration rate (using the Modification of Diet in Renal Disease study equation or the Chronic Kidney Disease Epidemiology Collaboration equation) of <60 mL/min/1.73m² at screening, repeatable once during the screening period;
History of acute hypersensitivity and/or anaphylaxis to protein therapeutics, components of formulation or allergies that could represent a substantial risk to the subject;
Participation in any clinical research study evaluating another investigational drug or therapy within 90 days or at least 5 half-lives (whichever is longer) for an investigational biologic drug, or at least 30 days for other investigational products, or within 6 months for immunotherapy prior to the screening visit of the current trial;
Sexually active men who are unwilling to use the following forms of medically acceptable birth control during the study drug treatment period through the EOS visit: vasectomy with medical assessment of surgical success OR consistent use of a condom; sperm donation is prohibited through the EOS visit;
Pregnant or breastfeeding women;
Women of childbearing potential (WOCBP, women who are fertile following menarche until becoming postmenopausal (no menses for 12 months without an alternative medical cause), unless permanently sterile, with permanent sterilization methods including hysterectomy, bilateral salpingectomy, and bilateral oophorectomy) who are unwilling to practice highly effective contraception prior to the initial dose/start of the first treatment, during the study, and through the EOS visit; highly effective contraceptive measures include: i) stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening, ii) intrauterine device; intrauterine hormone releasing system, iii) bilateral tubal ligation, iv) vasectomized partner (provided that the male vasectomized partner is the sole sexual partner of the WOCBP study participant and that the vasectomized partner has obtained medical assessment of surgical success for the procedure), and/or v) sexual abstinence (considered a highly effective method only if defined as refraining from heterosexual intercourse during the entire period of risk associated with the study drugs; the reliability of sexual abstinence needs to be evaluated in relation to the duration of the clinical trial and the preferred and usual lifestyle of the subject; periodic abstinence (calendar, symptothermal, post-ovulation methods), withdrawal (coitus interruptus), spermicides only, and lactational amenorrhea method are not acceptable methods of contraception; and female condom and male condom should not be used together).

### Study Treatments

REGN5381 or placebo will be dosed as a single fixed escalating IV dose as follows:
REGN5381 drug product is supplied as a 12.5 mg lyophilized powder in a sterile single use vial
Placebo to match REGN5381 is supplied as a lyophilized powder, in a sterile single use vial

Part A subjects will receive either REGN5381 IV or matching placebo IV as a single dose administered via IV infusion at a rate of 20 mL/hr using a syringe pump. Anticipated dose levels for Part A will include 0.3 mg IV up to a maximum dose of 10 mg IV.

Part B subjects will receive either REGN5381 IV or matching placebo IV as a single dose at dose level(s) administered via IV infusion at a rate of 20 mL/hr using a syringe pump. Dose level(s) in Part B will not exceed the doses tested in Part A and will be based on review of Part A data. A commercially-available crystalloid fluid bolus, 500 to 1,000 cc will be used in Part B.

Safety and tolerability data and PK/PD data from the initial cohorts are reviewed during the conduct of the study. The dose for subsequent cohorts may be adjusted based on emerging data from prior cohorts. Subjects will not receive doses higher than those specified in the protocol but may receive a lower dose or a dose previously administered to another cohort to confirm safety and tolerability and/or to further evaluate PD effects.

### Study Procedures

Blood pressure will be measured through inpatient monitoring and outpatient follow-up periods. Blood pressure will be assessed using two separate methods: an automated oscillometric upper arm cuff and a non-invasive finger cuff. The automated oscillometric BP measurement is used to assess BP for inclusion/exclusion and as a safety measure throughout the study at each visit. The finger cuff system collects BP data using a combination of photoplethysmography and a volume clamp method to continuously capture pulse wave data for non-invasive hemodynamic monitoring during the inpatient treatment/observation period. Continuous BP measurements will allow for precise PD assessment for the first 72 hours after dose administration. All oscillometric BP and HR measurements referred to in this study should be taken as 4 sequential measurements taken no sooner than approximately 1 minute apart. The median BP and HR measurement of the last 3 measurements should be used as the value for that time point. The subject's arm (left versus right) used for each assessment should be recorded.

Two-lead HR telemetry will be assessed for the duration of inpatient treatment/observation and extended safety inpatient monitoring.

Diet and water intake: subjects will be maintained on a fixed sodium diet for the length of the inpatient monitoring period. Subjects will be allowed approximately 3000 mg of sodium daily across all meals and snacks. Subjects will be maintained on a fixed water intake on days −1 and 1. The volume of oral fluid intake and urine voids will be recorded in 6-hour intervals. Prior to starting urine collection on day −1, subjects will be asked to empty their bladder. The start of urine collection and fluid volume restriction should match the start of dosing on day 1 (±2 hr.). For each interval, start/stop times are recorded, volume fluid in and volume fluid out are recorded. On each day −1 and day 1 the volume for fluid intake is fixed to approximately 2.5 L.

Part B fluid bolus challenge: a single crystalloid fluid bolus of 500 to 1,000 cc (via IV infusion over approximately 15 minutes) will be administered between 2 to 48 hours after study drug administration, to be determined based on PK/PD of Part A.

Laboratory testing: samples collected will be tested for blood chemistry (Sodium, Potassium, Chloride, Bicarbonate, Calcium, Glucose, Albumin, Total protein (serum), Creatinine, Blood urea nitrogen (BUN)/urea, Aspartate aminotransferase (AST), Alanine aminotransferase (ALT), Alkaline phosphatase, Lactate dehydrogenase (LDH), total bilirubin, triglycerides, uric acid, Creatine phosphokinase (CPK), Magnesium), hematology (hemoglobin, hematocrit, red blood cells (RBC), white blood cells (WBC), red cell indices, platelet count, differential (neutrophils, lymphocytes, monocytes, basophils, eosinophils)), and urinalysis (dipstick (bilirubin, pH, specific gravity, ketones, protein), urinobilogen, nitrite, RBC heme, glucose, microscopic (RBC count, WBC count, urine-formed elements)). Other laboratory tests include alcohol/drug, pregnancy, HIV, HBV, HCV, TSH, lipid panel, HbA1c, FSH, PD assessment, drug concentration, immunogenicity, and exploratory biomarkers.

Pharmacodynamic and explanatory biomarker procedures: research assessments will be performed to explore how REGN5381 may modify NPR1 signaling in healthy and hypertensive but otherwise healthy adults. In particular, the role of NPR1 agonism and the effect of REGN5381 on CGMP, natriuresis/diuresis, and cardiac function will be explored.

Additional physiologic measurements include 24-hour urine collection for assessment of diuresis and natriuresis. The natriuretic peptides and NPR1 agonists (e.g., nesiritide) have important effects on sodium and blood volume homeostasis. Nonclinical studies have not demonstrated effects of REGN5381 on sodium or water balance; however, it is important to test the effects in humans. For these reasons, subjects will be placed on a fixed sodium diet during inpatient monitoring, with fixed fluid intake on study days −1 and 1 and will have total ins and outs recorded for each 24 hour period. These data will provide additional data regarding the mechanisms of action of REGN5381 in healthy volunteers. Biomarker samples will be collected (prior to and after drug administration) at specified time points during the inpatient monitoring and outpatient follow-up periods.

Pharmacodynamic marker/biomarker measurements will be performed to determine effects on biomarkers of NPR1 activity, cardiac and renal function, or relevant physiological and pathogenic processes. The biomarkers studied are believed to be relevant to the pathophysiology of the indication, evidence of target engagement, mechanism of action of REGN5381 and/or possible early signs of toxicity. Biomarker measurements will provide additional information regarding the safety and efficacy of REGN5381. These will include but are not limited to biomarkers of cardiac injury (cardiac troponin).
Cardiac troponin plasma (high-sensitivity cardiac troponin-T (hs-cTnT) is a highly sensitive biomarker of cardiac ischemia in response to cardiac activity);
cGMP (plasma and urine cGMP are highly proximal measures of NPR1 activity in response to engagement by NPR1 agonists);
Sodium (urine sodium measures the excretion of sodium by the kidneys; increases in naturesis (or excretion of sodium in the urine) have been observed with engagement of NPR1; modulation of urine sodium by REGN5381 over time is an exploratory measure in this study; results from these analyses will be reported in the CSR);
renin (plasma renin measurements enable study of the impact of REGN5381 on other endogenous natriuretic hormones);
aldosterone (measurement of aldosterone at baseline and modulation of aldosterone over time are exploratory measures in this study; results from these analyses will be reported in the CSR);
NT-proBNP (plasma NT-proBNP is a biomarker that enables study of the impact of REGN5381 agonism of NPR1 on one of its measurable endogenous agonists);
Pulse wave analysis (pulse wave analysis allows for advanced hemodynamic parameters and continuous noninvasive BP from a finger cuff; continuous advanced hemodynamic parameters include: SBP, DBP, MAP, cardiac output, SV, SVV, dP/dt, and SVR; in certain clinical settings, an increase in SVV occurs with a reduction in venous pressures; derivative of BP over time in the pulse waveform can be used to approximate cardiac contractility under certain circumstances; systemic vascular resistance is calculated based on SBPs and SV; these data will provide information on the hemodynamic response to REGN5381 in healthy volunteers as well as those with hypertension); and
Pharmacogenomic analysis (whole blood samples can be collected for DNA extraction, to identify genomic associations with clinical or biomarker response to REGN5381, other heart failure, hypertension, and/or chronic kidney disease-related clinical outcome measures and possible AEs).

### Statistical Plan

Statistical analysis sets include i) Efficacy Analysis Sets; ii) Safety Analysis Set (SAF, including all randomized subjects who received any study drug; it is based on the treatment received (as treated); treatment compliance/administration and all clinical safety variables will be analyzed using the SAF); iii) Pharmacokinetic Analysis Sets (PK analysis population includes all subjects who received any study drug and who had at least 1 non-missing result following the first dose of study drug); and iv) Immunogenicity Analysis Sets (ADA analysis set, including all subjects who received study drug and had at least 1 non-missing ADA result following the first study dose).

Statistical methods: there are no formal primary efficacy analyses in this study. For continuous variables, descriptive statistics will include the following information: the number of subjects reflected in the calculation (n), mean, SD, Q1, median, Q3, minimum, and maximum. Plots of the values over time, as well as the change or percent change over time, will be provided. For categorical or ordinal data, frequencies and percentages will be displayed for each category. Unless otherwise specified, subjects on placebo will be pooled across cohorts within each of Part A and Part B.

### Pharmacokinetics

Analysis of drug concentration data: concentrations of total REGN5381 over time will be measured. Pharmacokinetic parameters may include, but are not limited to, the following (and their-dose-normalized versions, if applicable): AUCₗₐₛₜ (area under curve (AUC) computed from time zero to the time of the last positive concentration), AUC_{inf} (AUC from time zero extrapolated to infinity), Cₘₐₓ (peak concentration), Tₘₐₓ (time of peak concentration), Tₗₐₛₜ (time of the last positive (quantifiable) concentration), and CL (clearance). The concentrations of total REGN5381 over time and selected PK parameters will be summarized by descriptive statistics for each cohort for the purpose of estimating exposures in these groups. This descriptive statistical assessment will include the geometric means and may include ratios of the geometric means for selected PK parameters, as deemed appropriate.

Analysis of immunogenicity data: immunogenicity will be characterized by the ADA response observed: i) Pre-existing immunoreactivity (defined as a positive ADA assay response at baseline with all post-dose ADA results negative, or a positive assay response at baseline with all post-dose ADA assay responses less than 9-fold over baseline titer levels); ii) Treatment-emergent ADA response (defined as any post-dose positive ADA assay response when the baseline results are negative); iii) Treatment-boosted ADA response (defined as any post-dose positive ADA assay response that is 9-fold over baseline titer levels when the baseline is positive in the ADA assay); iv) Maximum ADA Titer values (Low (titer<1,000)/Moderate (1,000≤titer≤10,000)/High (titer>10,000)). Listings of pre-existing, treatment-boosted, and treatment-emergent ADA responses, ADA titers presented by subject, time point, and dose cohort/group will be provided. Incidence of treatment emergent ADA will be assessed as absolute occurrence (N) and percent of subjects (%), grouped by study cohorts and ADA titer level. Plots of drug concentrations will be examined and the influence of ADAs on individual PK profiles evaluated. Assessment of impact of ADA on safety and efficacy may be provided.

Analysis of pharmacodynamic and exploratory biomarker data: the PD biomarker population will consist of all subjects in the PK analysis set with at least 1 evaluable biomarker measurement at baseline and at least at 1 post-baseline time point. For biomarkers including plasma and urine cGMP, plasma renin levels, plasma aldosterone levels, plasma NT-proBNP, and hs-cTnT, the following descriptive data will be generated: raw data at baseline, by treatment group, and overall. Biomarkers measured post treatment, will be summarized over time, change and/or percent change from baseline to each scheduled assessment time will be summarized by treatment with descriptive statistics. In addition, for each visit, the mean concentrations and mean percent change from baseline will be generated.

As to the assessment of the effect of single IV doses of REGN5381 on BP and HR in normotensive and otherwise healthy hypertensive adults and of the effects of REGN5381 on cardiac SV, each scheduled assessment and change from baseline in SBP, DBP, MAP, PP and HR over time will be summarized with descriptive statistics, along with 2-sided 90% confidence interval, by dose cohort, by treatment group. Maximum time-matched change from baseline in SBP, DBP, MAP, PP and cardiac SV across the first 24 hours post-dose will be reported with local regression or moving average by dose cohort, by treatment group. Mean and/or percent change from baseline in SBP, DBP and MAP will be plotted over time by dose cohort, by treatment group. Dose-response for reduction from baseline in SBP, DBP, SV, and PP will be evaluated quantitatively with appropriate statistical models.

Analyses of other exploratory endpoints will also be done.

### Status and Preliminary Results

In Cohorts 1-4 of Part A, 32 subjects (25 males, 7 females, ages 20 to 51) were randomized as planned (REGN5381 vs placebo in a 6:2 ratio), and no subjects were discontinued from the study. As part of dose escalation, a blinded review of safety data was performed after the EOS visit of Cohort 4, when all subjects had completed 21 days of follow-up after a single dose of study drug (REGN5381 or placebo). Treatment with study drug was generally well-tolerated when administered via the IV route at a single dose up to 10 mg. There were no treatment interruptions or discontinuations. No serious treatment-emergent adverse events (TEAEs), severe TEAEs, or deaths were reported. Mild TEAEs were reported by 13 subjects (41%) and moderate TEAEs were reported by one subject (3%). The most frequent TEAEs were hematuria and pyuria on routine urinalysis. These TEAEs occurred in 5 asymptomatic individuals, each with a single abnormal test result with no obvious correlation between dose cohorts. The one subject with moderate TEAEs experienced nausea and vomiting at the time of infusion on day 1. Symptomatic hypotension and tachycardia were not observed. In all 4 cohorts, there were no clinically significant or dose-dependent treatment-emergent changes in hematologic and chemistry parameters. There were no clinically significant abnormalities or dose-dependent shifts from baseline in vital signs and in any ECG parameter (i.e., ventricular rate, PR, QRS, QT and RR intervals). All subjects across all four cohorts met inpatient discharge criteria at 72 hours.

Enrollment of mildly hypertensive patients has begun, and results to-date are in line with those observed in the healthy volunteers.

### Example 6. REGN5381 Induces Sustained and Well-tolerated Modulation of Systemic Hemodynamics in Healthy Patients

### Background

Natriuretic peptide receptor 1 (NPR1) is a membrane-bound guanylate cyclase; NPR1 agonism alters blood pressure (BP) via cGMP-mediated effects on intravascular volume, vasorelaxation, natriuresis, and diuresis. NPR1 is activated by natriuretic peptides (NPs); NPR1 and NPs are therapeutic targets for patients with heart failure (HF) due to their role in fluid homeostasis and sodium balance. The generation, pre-clinical characterization, and first-in-human (FIH) evaluation of REGN5381, an agonist antibody to NPR1, is reported in the instant study.

### Methods

REGN5381 was isolated using the VelocImmune® technology platform. In vivo pre-clinical pharmacology experiments were conducted in mice and cynomolgus monkeys. In an ongoing Phase 1, double-blind, placebo-controlled, two-part single-ascending dose study designed to assess safety, tolerability, and pharmacokinetics/pharmacodynamics of REGN5381 (Example 5; NCT04506645), 32 healthy adults were randomized 6:2 to single-dose IV REGN5381 (0.3, 1, 3 or 10 mg) or IV placebo.

### Results

Persistent, dose-dependent systolic BP (SBP) lowering (˜12 mm Hg) was observed in normotensive NPR1-humanized mice following REGN5381 dosing, with additive effects when combined with SOC oral therapeutics. REGN5381 25 mg/kg induced sustained SBP lowering in hypertensive NPR1-humanized mice (˜30 mm Hg). In normotensive cynomolgus monkeys, REGN5381 reduced SBP (10-15 mm Hg), with high doses (25 mg/kg SC and IV) having modest effects that persisted for the duration of the study (56 days). In the ongoing FIH study, REGN5381 reduced SBP by 6-9 mm Hg 12-24 hours post-dose, with no change in urine output across all dose cohorts. The reduction in SBP was associated with an increase in plasma cGMP. No serious adverse events were reported, and the most common study drug related treatment-emergent adverse event (TEAE) was postural dizziness, reported in 3 subjects across 3 different dose cohorts. Other study drug-related TEAEs included palpitations and headache, reported in 2 subjects each.

### Conclusion

Preclinical and first-in-human results indicate that the NPR1 agonist, REGN5381, provides sustained hemodynamic effects, with no evidence of adverse hypotension in normotensive healthy volunteers.

### Example 7. REGN5381 In-human Evaluation

A phase 1, double-blind, placebo-controlled, two-part single-ascending dose study was designed to assess safety, tolerability, and pharmacodynamics/pharmacokinetics of REGN5381 in healthy adults (18-55 years of age).

Participants were randomized 6:2 to receive single-dose intravenous REGN5381 (0.3, 1, 3, 10, 30, or 100 mg) or intravenous placebo. Following study drug administration on day 1, participants remained in the clinic through day 4 to allow for careful monitoring of hemodynamics. On day 4, participants were assessed for safety prior to discharge. Study duration was based on predicted blood levels of REGN5381 concentrations in serum across all dose cohorts; the end of study visit was conducted no earlier than 21 days following the administration of study drug. The primary endpoint in the instant study was the type, incidence, and severity of TEAEs following a single intravenous dose administrations of REGN5381 or placebo over time.

Table 11, below, presents data on mean change from baseline in pulse pressure assessed via pulse wave analysis according to intravenous treatment group (pooled placebo, REGN5381 10 mg, REGN5381 30 mg, or REGN5381 100 mg). Mean change from baseline in pulse pressure is presented as an average over 24 hours.

TABLE 11
Mean change in pulse pressure (PP)

| | | | | Standard |
|---|---|---|---|---|
| | Study | Number of | Change from | Error |
| Treatment Group | Day | subjects | baseline in PP | for PP |
| Normotensive | Day 1 | 12 | 1 | 1.2 |
| Pooled Placebo | | | | |
| (N = 12) | | | | |
| Normotensive | Day 2 | 12 | −1.4 | 1.99 |
| Pooled Placebo | | | | |
| (N = 12) | | | | |
| Normotensive | Day 3 | 12 | 0.4 | 1.56 |
| Pooled Placebo | | | | |
| (N = 12) | | | | |
| 10 mg IV (N = 6) | Day 1 | 6 | −0.54 | 2.65 |
| 10 mg IV (N = 6) | Day 2 | 6 | −1.95 | 3.5 |
| 10 mg IV (N = 6) | Day 3 | 6 | −2.68 | 2.92 |
| 30 mg IV (N = 6) | Day 1 | 6 | −1.67 | 1.33 |
| 30 mg IV (N = 6) | Day 2 | 6 | 0.69 | 2.62 |
| 30 mg IV (N = 6) | Day 3 | 6 | 2.39 | 3.39 |
| 100 mg IV (N = 6) | Day 1 | 6 | −4.05 | 1.88 |
| 100 mg IV (N = 6) | Day 2 | 6 | −9.6 | 2.26 |
| 100 mg IV (N = 6) | Day 3 | 6 | −6.72 | 2.1 |

REGN 100 mg IV showed a large and significant reduction in pulse pressure vs. placebo after a single administration over the 3-day course of monitoring.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method of treating a disease associated with altered hemodynamics in a subject, comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

2. The method of claim 1, wherein the disease is selected from the group consisting of heart failure, hypertension, and chronic kidney disease.

3. A method of reducing blood pressure in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

4. The method of claim 3, wherein the blood pressure is selected from the group consisting of systolic blood pressure, diastolic blood pressure, mean arterial pressure, and pulse pressure.

5. A method of effecting a hemodynamic change in a subject, the method comprising: (i) selecting a subject with systolic blood pressure (SBP) between 100 mm Hg and 140 mm Hg or between 135 mm Hg and 160 mm Hg; and (ii) administering to the subject a therapeutically effective amount of an antibody or antigen-binding fragment thereof that specifically binds natriuretic peptide receptor 1 (NPR1).

6. The method of claim 5, wherein the hemodynamic change is a reduction in venous pressure, left ventricular end-diastolic pressure (LVEDP), and/or arterial pulse pressure (PP).

7. The method of claim 6, wherein the reduction in venous pressure is a reduction in central venous pressure (CVP).

8. The method of claim 5, wherein urine output and systemic organ perfusion are not affected by the administration.

9. The method of claim 1, wherein the heart rate (HR) of the subject increases.

10. The method of claim 1, wherein the antibody or antigen-binding fragment thereof comprises three heavy chain complementarity determining regions (CDRs) (HCDR1, HCDR2 and HCDR3) contained within a heavy chain variable region (HCVR) comprising the amino acid of SEQ ID NO: 1; and three light chain CDRs (LCDR1, LCDR2 and LCDR3) contained within a light chain variable region (LCVR) comprising the amino acid sequence of SEQ ID NO:2.

11. The method of claim 1, wherein the antibody or antigen-binding fragment thereof comprises three heavy chain complementarity determining regions (CDRs) (HCDR1, HCDR2 and HCDR3) and three light chain CDRs (LCDR1, LCDR2 and LCDR3), wherein HCDR1 has the amino acid sequence of SEQ ID NO:3, HCDR2 has the amino acid sequence of SEQ ID NO:4, HCDR3 has the amino acid sequence of SEQ ID NO:5, LCDR1 has the amino acid sequence of SEQ ID NO:6, LCDR2 has the amino acid sequence of VAS, and LCDR3 has the amino acid sequence of SEQ ID NO:8.

12. The method of claim 1, wherein the antibody or antigen-binding fragment thereof comprises a heavy chain variable region (HCVR) comprising the amino acid of SEQ ID NO:1 and a light chain variable region (LCVR) comprising the amino acid sequence of SEQ ID NO:2.

13. The method of claim 1, wherein the antibody comprises a heavy chain and a light chain, wherein the heavy chain comprises an amino acid sequence of SEQ ID NO: 9.

14. The method of claim 1, wherein the antibody comprises a heavy chain and a light chain, wherein the light chain comprises an amino acid sequence of SEQ ID NO: 10.

15. The method of claim 1, wherein the antibody comprises a heavy chain and a light chain, wherein the heavy chain comprises an amino acid sequence of SEQ ID NO: 9 and the light chain comprises an amino acid sequence of SEQ ID NO: 10.

16. The method of claim 1, wherein the antibody or antigen-binding fragment thereof is administered at a low dose.

17. The method of claim 1, wherein the antibody or antigen-binding fragment thereof is administered at a dose of about 0.031 to about 25 mg/kg of the body weight of the subject.

18. The method of claim 1, wherein the antibody or antigen-binding fragment thereof is administered at a dose of about 1 mg to about 200 mg.

19. The method of claim 1, wherein the antibody or antigen-binding fragment thereof is administered intravenously, subcutaneously, intradermally, intraperitoneally, or intramuscularly to the subject.

20. The method of claim 1, wherein the antibody or antigen-binding fragment thereof is administered to the subject as a single dose.

21. The method of claim 1, further comprising administering an additional therapeutic agent to the subject.

22. The method of claim 21, wherein the second therapeutic agent is administered concomitantly with or separately from the antibody or antigen-binding fragment thereof.

23. The method of claim 1, wherein the subject is hypertensive.

24. The method of claim 23, wherein the subject is mildly hypertensive.

25. The method of claim 1, wherein the subject is human.

26. A pharmaceutical composition for use in treating a disease associated with altered hemodynamics in a subject, comprising a therapeutically effective amount of an antibody or antigen-binding fragment thereof that specifically binds natriuretic peptide receptor 1 (NPR1) and a pharmaceutically acceptable carrier or diluent.

27. A pharmaceutical composition for use in reducing blood pressure in a subject, comprising a therapeutically effective amount of an antibody or antigen-binding fragment thereof that specifically binds NPR1 and a pharmaceutically acceptable carrier or diluent.

28. A pharmaceutical composition for use in effecting hemodynamic changes in a subject, comprising a therapeutically effective amount of an antibody or antigen-binding fragment thereof that specifically binds natriuretic peptide receptor 1 (NPR1) and a pharmaceutically acceptable carrier or diluent.


SEQUENCE LISTING (10 sequences)

SEQ ID NO: 1 - protein, 124 aa, synthetic construct
QVQLVQSGAEVKKPGASVTVSCKASGYTFTDYYMHWVRQAPGQGLEWMGWIKPNSGGTNSAQRFQGRITMTWDTSISTAYMELSRLRSDDTAVYYCSRGGPVMNYYYYYGMDVWGQGTTVTVSS

SEQ ID NO: 2 - protein, 106 aa, synthetic construct
NIQMTQSPSSLSASVGDRVTITCRASQSIDSYLNWYQQKPGKAPKLLIYVASSLQSGVPSRFSGSGSGKDFTLTISSLQPEDFATYYCQQSYSIPTFGQGTRLEIK

SEQ ID NO: 3 - protein, 8 aa, synthetic construct
GYTFTDYY

SEQ ID NO: 4 - protein, 8 aa, synthetic construct
IKPNSGGT

SEQ ID NO: 5 - protein, 17 aa, synthetic construct
SRGGPVMNYYYYYGMDV

SEQ ID NO: 6 - protein, 6 aa, synthetic construct
QSIDSY

SEQ ID NO: 7 - skipped

SEQ ID NO: 8 - protein, 8 aa, synthetic construct
QQSYSIPT

SEQ ID NO: 9 - protein, 451 aa, synthetic construct
QVQLVQSGAEVKKPGASVTVSCKASGYTFTDYYMHWVRQAPGQGLEWMGWIKPNSGGTNSAQRFQGRITMTWDTSISTAYMELSRLRSDDTAVYYCSRGGPVMNYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 213 aa, synthetic construct
NIQMTQSPSSLSASVGDRVTITCRASQSIDSYLNWYQQKPGKAPKLLIYVASSLQSGVPSRFSGSGSGKDFTLTISSLQPEDFATYYCQQSYSIPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC